(19) United States
(12) Patent Application Publication
(10) Pub. No.: US 2024/0108275 A1
(43) Pub. Date: Apr. 4, 2024

(54) ANALYSIS SYSTEM, ANALYSIS METHOD, ANALYTICAL DATA ACQUISITION DEVICE, AND ANALYSIS PROGRAM

(71) Applicant: Saitama Medical University, Saitama (JP)

(72) Inventor: Susumu SHINDO, Saitama (JP)

(21) Appl. No.: 18/257,144

(22) PCT Filed: Dec. 22, 2021
(86) PCT No.: PCT/JP2021/047703

§ 371 Date: Jun. 13, 2023

(30) Foreign Application Priority Data
Dec. 23, 2020 (JP) 2020-213471

(51) Int. Cl. A61B 5/00; A61B 3/113; A61B 5/11

(52) U.S. Cl. CPC A61B 5/4023; A61B 3/113; A61B 5/1114; A61B 5/746

(57) ABSTRACT

To provide an analysis system for analysis of semicircular canal functions with rotational stimuli. The analysis system includes a vestibulo ocular reflex data-acquiring unit configured to acquire first vestibulo ocular reflex data and second vestibulo ocular reflex data. The first vestibulo ocular reflex data is acquired by dividing a rotation angle owing to a vestibulo ocular reflex with a rotation angle of a head position. The second vestibulo ocular reflex data is acquired by subtracting at least a rotation angle owing to catch-up saccades (CUS) from the rotation angle of the head position to determine a residual rotation angle, followed by dividing the residual rotation angle with the rotation angle of the head position. The analysis system is configured to analyze semicircular canal functions based on the second vestibulo ocular reflex data, or based on both the first vestibulo ocular reflex data and the second vestibulo ocular reflex data.

### TECHNICAL FIELD

The present invention relates to an analysis system, an analysis method, an analytical data acquisition device, and an analysis program.

### BACKGROUND ART

The video Head Impulse Test (may be referred to as “vHIT” hereinafter) is one of the tests for semicircular canal functions using rotational stimuli, which was developed in 2009. The vHIT has rapidly become popular and actively used all over the world because the vHIT enables examination of semicircular canal functions according to a physiological stimulation method in comparison with a conventional caloric test using thermal stimuli, the vHIT can study vertical semicircular canal functions in addition to horizontal semicircular canal functions, and a test is easily performed.

The vHIT utilizes a vestibulo ocular reflex (may be referred to as “VOR” hereinafter) triggered when the head of a subject (patient) is turned rapidly, and measures an angular velocity of a head position and angular velocities of eye positions during the VOR. Evaluation items of the vHIT include VOR_{gain} and catch-up saccades (may be referred to as “CUS” hereinafter) triggered when the VOR_{gain} is deficient. Among the above-mentioned evaluation items, the VOR_{gain} is important to quantify a semicircular canal function test.

According to a method of directly calculating the VOR_{gain} from a ratio between angles that are an integral of angular velocities of the head position and an integral of angular velocities of an eye position during VOR, respectively, or an angular velocity ratio between the head position and eye position during a period from onset of VOR to the predetermined time (may be referred to as a “direct method” hereinafter), the VOR_{gain} of a healthy individual is about 1, but VOR_{gain} of a patient having impaired semicircular canal functions declines. When the VOR_{gain} becomes approximately less than 0.8, the patient cannot gaze at a target, thus the CUS is triggered.

In the vHIT, slippage-induced artifacts and noise artifacts may occur due to characteristics of the test using rotational stimuli, where the slippage-induced artifacts are caused by slippage of goggles set on a head during turning motions, and the noise artifacts are caused as the eye positions are not accurately measured. In the above-described cases, there is a problem such that the VOR_{gain} does not indicate an appropriate value in the direct method because the VOR_{gain} is noticeably affected by the artifacts (see, for example NPL 1).

Moreover, the VOR_{gain} of a patient having impaired semicircular canal functions may decline as an angular velocity during the rotational stimulus increases. However, the current vHIT does not take the influence of the angular velocity into consideration, which may be problematic.

As increased use of the vHIT is expected in the coming years, it is unavoidable to improve the test accuracy for the VOR_{gain}.

### CITATION LIST

### Non-Patent Literature

[NPL 1] “Mutual evaluation of Caloric Test and video Head Impulse Test,” Susumu SHINDO et el., Equilibrium Res. Vol. 74(6) 541-551 2015)

### SUMMARY OF INVENTION

### Technical Problem

The present invention aims to provide an analysis system, an analysis method, an analytical data acquisition device, and an analysis program, which are not influenced by artifacts induced by slippage and the like, significantly improve accuracy of tests, and can accurately and precisely analyze semicircular canal functions.

### Solution to Problem

The means for solving the above-described problems are as follows.

<1> An analysis system for analysis of semicircular canal functions with rotational stimuli, the analysis system including:
a vestibulo ocular reflex data-acquiring unit configured to acquire first vestibulo ocular reflex data and second vestibulo ocular reflex data, where the first vestibulo ocular reflex data is acquired by dividing a rotation angle owing to a vestibulo ocular reflex with a rotation angle of a head position, and the second vestibulo ocular reflex data is acquired by subtracting at least a rotation angle owing to a catch-up saccade (CUS) from the rotation angle of the head position to determine a residual rotation angle, followed by dividing the residual rotation angle with the rotation angle of the head position,
wherein the analysis system is configured to analyze semicircular canal functions based on the second vestibulo ocular reflex data, or based on both the first vestibulo ocular reflex data and the second vestibulo ocular reflex data.

<2> The analysis system according to <1>,
wherein the second vestibulo ocular reflex data is a value of VOR_{gain (I)} determined based on angular velocity data of a head of a subject and angular velocity data of eye movements of the subject, which are acquired by an angular velocity data-acquiring unit, according to the following equation:

VOR_{gain(I)}=(A−C)/A
wherein the angular velocity data-acquiring unit is set on the head of the subject and is configured to acquire the angular velocity data of the head and the angular velocity data of the eye movements when the head is turned, and
wherein, in the above equation, A is an integral of the angular velocity data of the head and C is an integral of the angular velocity data of the eyes of the subject owing to catch-up saccades (CUS) during a time when the head is turned.

<3> The analysis system according to <2>,
wherein the first vestibulo ocular reflex data is a value of VOR_{gain (D)} determined based on the angular velocity data of the head and the angular velocity data of the eye movements acquired by the angular velocity data-acquiring unit according to the following equation:

VOR_{gain(D)}=B/A
where A is the integral of the angular velocity data of the head, and B is an integral of the angular velocity data of the eye movements during the time when the head is turned.

<4> An analysis system for analysis of semicircular canal functions with rotational stimuli, the analysis system including:
an angular velocity data-acquiring unit that is set on a head of a subject and is configured to acquire angular velocity data of the head, and angular velocity data of eye movements of the subject when the head is turned; and
an analysis unit configured to determine a value of VOR_{gain (I)} based on the angular velocity data of the head and the angular velocity data of the eye movements, which are acquired by the angular velocity data-acquiring unit, according to the following equation:

VOR_{gain(I)}=(A−C)/A
where A is an integral of the angular velocity data of the head and C is an integral of the angular velocity data of the eyes of the subject owing to catch-up saccades (CUS) during a time when the head is turned, and
to analyze semicircular canal functions of the subject based on the value of the VOR_{gain (I)}.

<5> The analysis system according to any one of <2> to <4>, wherein a data value of the A is a sum of a plurality of integrals of multiple angular velocity data of the head, and a data value of the C is a sum of a plurality of integrals of multiple angular velocity data of the eyes of the subject owing to the catch-up saccades (CUS).

<6> The analysis system according to any one of <2> to <5>, wherein the analysis system is configured to acquire a plurality of values of the VOR_{gain (I)}, determine a line of best fit from the acquired values of the VOR_{gain (I)}, and determine a value of the VOR_{gain (I)} at a certain angular velocity from the line of best fit.

<7> The analysis system according to any one of <2> to <6>, wherein the analysis system is configured to acquire a plurality of values of the VOR_{gain (I)}, determine a line of best fit from the acquired values of the VOR_{gain (I)}, and determine dispersion of the acquired values of the VOR_{gain (I)} from the line of best fit.

<8> The analysis system according to any one of <2> to <7>, wherein the analysis system is configured to determine values of VOR_{gain (D)} based on the angular velocity data of the head and the angular velocity data of the eye movements, which are acquired by the angular velocity data-acquiring unit, according to the following equation:

VOR_{gain(D)}=B/A
where A is an integral of the angular velocity data of the head and B is an integral of the angular velocity data of the eye movements during the time when the head is turned, and the analysis system is configured to calculate an arithmetic mean and standard deviation of differences each between the value of the VOR_{gain (D)} and the value of the VOR_{gain (I)}.

<9> The analysis system according to <8>, further including:
a warning unit configured to display the values of VOR_{gain (D)}, the values of VOR_{gain (I)}, and the arithmetic mean and standard deviation of the differences each between the value of the VOR_{gain (D)} and the value of the VOR_{gain (I)}, and to issue a warning when the values exceed standard reference ranges.

<10> The analysis system according to any one of <2> to <9>, wherein the angular velocity data-acquiring unit includes a sensor and a camera, where the sensor is configured to collect angular velocity data associated with rotational movements of the head of the subject, and the camera is configured to capture the eye movements of the subject.

<11> An analytical data acquisition device including:
a sensor configured to collect angular velocity data associated with rotational movements of a head of a subject; and
a camera configured to capture eye movements of the subject,
   wherein the analytical data acquisition device is used to analyze semicircular canal functions with rotational stimuli.

<12> An analysis method for analysis of semicircular canal functions with rotational stimuli, the analysis method including:
a vestibulo ocular reflex data-acquiring process that includes
   acquiring first vestibulo ocular reflex data and second vestibulo ocular reflex data,
where the first vestibulo ocular reflex data is acquired by dividing a rotation angle owing to a vestibulo ocular reflex with a rotation angle of a head position, and the second vestibulo ocular reflex data is acquired by subtracting at least a rotation angle owing to catch-up saccades (CUS) from the rotation angle of the head position to determine a residual rotation angle, followed by dividing the residual rotation angle with the rotation angle of the head position,
wherein the analysis method analyzes semicircular canal functions based on the second vestibulo ocular reflex data, or based on both the first vestibulo ocular reflex data and the second vestibulo ocular reflex data.

<13> An analysis method for analysis of semicircular canal functions with rotational stimuli, the analysis method including:
an angular velocity data-acquiring process that includes
   setting on a head of a subject, and acquiring angular velocity data of the head and angular velocity data of eye movements of the subject when the head is turned; and
an analysis process that includes
   determining a value of VOR_{gain (I)} based on the angular velocity data of the head and the angular velocity data of the eye movements, which are acquired in the angular velocity data-acquiring process, according to the following equation:

VOR_{gain(I)}=(A−C)/A
where A is an integral of angular velocity data of the head, and C is an integral of angular velocity data of the eyes of the subject owing to catch-up saccades (CUS) during a time when the head is turned, and
   analyzing semicircular canal functions based on the value of the VOR_{gain (I)}.

<14> An analysis program for analysis of semicircular canal functions with rotational stimuli, the analysis program causing a computer to execute processes of:
acquiring first vestibulo ocular reflex data and second vestibulo ocular reflex data, where the first vestibulo ocular reflex data is acquired by dividing a rotation angle owing to a vestibulo ocular reflex with a rotation angle of a head position, and the second vestibulo ocular reflex data is acquired by subtracting at least a rotation angle owing to catch-up saccades (CUS) from the rotation angle of the head position to determine a residual rotation angle, followed by dividing the residual rotation angle with the rotation angle of the head position; and
analyzing semicircular canal functions based on the second vestibulo ocular reflex data, or based on both the first vestibulo ocular reflex data and the second vestibulo ocular reflex data.

<15> An analysis program for analysis of semicircular canal functions with rotational stimuli, the analysis program causing a computer to execute processes of:
acquiring angular velocity data of a head of a subject and angular velocity data of eye movements of the subject when the head of the subject is turned;
determining VOR_{gain (I)} based on the angular velocity data of the head and the angular velocity data of the eye movements according to the following equation:

VOR_{gain(I)}=(A−C)/A
where A is an integral of the angular velocity data of the head and C is an integral of the angular velocity data of the eyes of the subject owing to catch-up saccades (CUS) during a time when the head is turned; and
analyzing semicircular canal functions based on the value of VOR_{gain (I)}.

### Advantageous Effects of Invention

According to the present invention, an analysis system, an analysis method, an analytical data acquisition device, and an analysis program, which are not influenced by artifacts induced by slippage and the like, significantly improve accuracy of tests, and can accurately and precisely analyze semicircular canal functions, can be provided.

### BRIEF DESCRIPTION OF DRAWINGS

FIG. 1 is a graph depicting a relationship between a time and angular velocity in the vHIT measurement.

FIG. 2A is a graph depicting a relationship between a time and angular velocity to explain a direct method where VOR_{gain} is directly determined by the vHIT measurement.

FIG. 2B is a graph depicting a relationship between a time and angular velocity to explain a direct method where VOR_{gain} is directly determined by the vHIT measurement.

FIG. 3 is a graph depicting a relationship between a time and angular velocity to explain an indirect method where VOR_{gain} is indirectly determined by the vHIT measurement.

FIG. 4 is a diagram depicting an example of the hardware configuration of the analysis system of the present invention.

FIG. 5 is a diagram depicting an example of the functional configuration of the analysis system of the present invention.

FIG. 6 is a flowchart depicting an example of a flow of processes of the analysis method of the present invention.

FIG. 7 is a graph depicting a relationship between a time and angular velocity determined by measuring a subject under the conditions without slippage-induced artifacts in Example 1.

FIG. 8 is a graph depicting a relationship between a time and angular velocity determined by measuring the subject with intentionally caused-slippage in Example 1.

FIG. 9A is a graph depicting a relationship between an angular velocity and VOR_{gain} of a healthy individual in Example 2.

FIG. 9B is a graph depicting a relationship between an angular velocity and VOR_{gain} of a patient having unilateral impairments of vestibular functions in Example 2.

FIG. 10 is a graph depicting a relationship between an angular velocity and VOR_{gain} of an example where VOR_{gain} changes depending on the angular velocity in Example 2.

FIG. 11 is a graph depicting a method of determining a line of best fit in Example 2 to determine VOR_{gain} at the predetermined angular velocity.

FIG. 12A is a graph depicting a relationship between an angular velocity and VOR_{gain} of an example without many artifacts and with substantially constant VOR_{gain} in Example 3.

FIG. 12B is a graph depicting a relationship between an angular velocity and VOR_{gain} of an example with many artifacts and with dispersed VOR_{gain} in Example 3.

FIG. 13A is a graph depicting a relationship between an angular velocity and VOR_{gain} of an example with many artifacts but with a small standard deviation in Example 3.

FIG. 13B is a graph depicting a relationship between an angular velocity and VOR_{gain} of an example without many artifacts and a small deviation in Example 3.

FIG. 14A is a graph depicting a relationship between an angular velocity and VOR_{gain}, when linear regression is performed on FIG. 13A of Example 3 according to the least squares method.

FIG. 14B is a graph depicting a relationship between an angular velocity and VOR_{gain}, when linear regression is performed on FIG. 13B of Example 3 according to the least squares method.

### DESCRIPTION OF EMBODIMENTS

### (Analysis System and Analysis Method)

In the first embodiment, the analysis system of the present invention is directed to an analysis system for analysis of semicircular canal functions with rotational stimuli. The analysis system of the first embodiment includes a vestibulo ocular reflex data-acquiring unit configured to acquire first vestibulo ocular reflex data and second vestibulo ocular reflex data, where the first vestibulo ocular reflex data is acquired by dividing a rotation angle owing to a vestibulo ocular reflex with a rotation angle of a head position, and the second vestibulo ocular reflex data is acquired by subtracting at least a rotation angle owing to catch-up saccades (CUS) from the rotation angle of the head position to determine a residual rotation angle, followed by dividing the residual rotation angle with the rotation angle of the head position. The analysis system is configured to analyze semicircular canal functions based on the second vestibulo ocular reflex data, or based on both the first vestibulo ocular reflex data and the second vestibulo ocular reflex data.

In the second embodiment, the analysis system of the present invention is directed to an analysis system for analysis of semicircular canal functions with rotational stimuli. The analysis system of the second embodiment includes an angular velocity data-acquiring unit and an analysis unit. The angular velocity data-acquiring unit is set on the head of a subject and is configured to acquire angular velocity data of the head and angular velocity data of eye movements of the subject when the head is turned. The analysis unit is configured to determine a value of VOR_{gain (I)} based on the angular velocity data of the head and the angular velocity data of the eye movements, which are acquired by the angular velocity data-acquiring unit, according to the following equation, and to analyze semicircular canal functions of the subject based on the value of the VOR_{gain (I)}.

VOR_{gain(I)}=(A−C)/A

In the above equation, A is an integral of the angular velocity data of the head and C is an integral of the angular velocity data of the eyes of the subject owing to catch-up saccades (CUS) during the time when the head is turned. The analysis system may further include other units as necessary.

In the first embodiment, the analysis method of the present invention is an analysis method for analysis of semicircular canal functions with rotational stimuli, and the analysis method includes a vestibulo ocular reflex data-acquiring process that includes acquiring first vestibulo ocular reflex data and second vestibulo ocular reflex data. The first vestibulo ocular reflex data is acquired by dividing a rotation angle owing to a vestibulo ocular reflex with a rotation angle of a head position. The second vestibulo ocular reflex data is acquired by subtracting at least a rotation angle owing to catch-up saccades (CUS) from the rotation angle of the head position to determine a residual rotation angle, followed by dividing the residual rotation angle with the rotation angle of the head position. The analysis method analyzes semicircular canal functions based on the second vestibulo ocular reflex data, or based on both the first vestibulo ocular reflex data and the second vestibulo ocular reflex data.

In the second embodiment, the analysis method of the present invention is an analysis method for analysis of semicircular canal functions with rotational stimuli, and the analysis method includes an angular velocity data-acquiring process and an analysis process. The angular velocity data-acquiring process includes setting on the head of a subject, and determining a value of VOR_{gain (I)} based on the angular velocity data of the head and the angular velocity data of the eye movements, which are acquired in the angular velocity data-acquiring process, according to the following equation, and analyzing semicircular canal functions based on the value of the VOR_{gain (I)}.

VOR_{gain(I)}=(A−C)/A

In the above equation, A is an integral of angular velocity data of the head, and C is an integral of angular velocity data of the eyes of the subject owing to catch-up saccades (CUS) during the time when the head is turned. The analysis method may further include other processes as necessary.

The analysis methods of the first and second embodiments of the present invention can be suitably performed by the analysis systems of the first and second embodiments of the present invention. The vestibulo ocular reflex data-acquiring process can be performed by the vestibulo ocular reflex data-acquiring unit. The angular velocity data-acquiring process can be performed by the angular velocity data-acquiring unit. The analysis process can be performed by the analysis unit. Other processes can be performed by other units.

The analysis systems and analysis methods of the first and second embodiments of the present invention are analysis systems and analysis methods for analysis of semicircular canal functions with rotational stimuli.

Since most of chordates including humans have three semicircular canals, the semicircular canals may be referred to as three semicircular canals. The three semicircular canals are organs for regulating a sense of balance (angular acceleration), and are the collective name for three semicircular ducts each in the shape of semicircular tube, and connected to the vestibular system of the inner ear.

As the method of testing semicircular canal functions with rotational stimuli, there are a head impulse test (HIT) and a video head impulse test (vHIT).

In the HIT, first, a tester sits to face a subject, and instructs the subject to continuously gaze at a target on the tester (tip of nose). Next, the tester holds the sides of the head of the subject with both hands, and turns the head of the subject to rotate the head with fast and small motions to give head impulse (HI). When HI is given to a healthy individual, such individual can continue to gaze at the target (tip of nose) owing to the action of vestibulo ocular reflex (VOR). If HI is given to a subject having impaired semicircular canal functions in the direction towards the side where the semicircular canal functions of the subject are impaired, however, VOR does not sufficiently function thus the subject cannot continuously gaze at the target, and catch-up saccades (CUS) appear approximately 200 msec after the onset of the impulse. In HIT, a case where 2 or more catch-up saccades (CUS) are observed with the naked eye when HI is applied three times each for the left and right sides, is determined as impaired semicircular canal functions.

The HIT has advantages such that it is not invasive for a patient compared to a caloric test using thermal stimuli, the test can be performed within a short-period of time, and the test can be performed with physiological stimulation. On the other hand, the HIT has problems such that a result may be subjectively biased because it is judged by the observation with the naked eye of the tester, it cannot detect a type of impaired semicircular canal functions where onset of CUS cannot be detected with the naked eye, and a quantitative evaluation of semicircular canal functions cannot be performed.

The vHIT is a test that has overcome the problems of the HIT, and requires a device specifically designed for vHIT to simultaneously record an angular velocity of a head position and an angular velocity of eye position during a test. Examples of a type of a device for vHIT where special goggles with build-in high-speed camera and sensor are designed to be set on the head of a subject, as the above-described device specifically designed for vHIT, include ICS Impulse manufactured by Natus Medical Incorporated, Eye See Cam manufactured by Interacoustics A/S, and the like.

### <Vestibulo Ocular Reflex Data-Acquiring Process and Vestibulo Ocular Reflex Data-Acquiring Unit>

The vestibulo ocular reflex data-acquiring process is a process that includes acquiring first vestibulo ocular reflex data and second vestibulo ocular reflex data. The first vestibulo ocular reflex data is acquired by dividing a rotation angle owing to a vestibulo ocular reflex with a rotation angle of a head position. The second vestibulo ocular reflex data is acquired by subtracting at least a rotation angle owing to catch-up saccades (CUS) from the rotation angle of the head position to determine a residual rotation angle, followed by dividing the residual rotation angle with the rotation angle of the head position. The vestibulo ocular reflex data-acquiring process is performed by the vestibulo ocular reflex data-acquiring unit.

In the analysis system and analysis method of the first embodiment of the present invention, semicircular canal functions are analyzed based on the second vestibulo ocular reflex data, or based on both the first vestibulo ocular reflex data and the second vestibulo ocular reflex data.

The second vestibulo ocular reflex data is data acquired by subtracting at least a rotation angle owing to catch-up saccades (CUS) from the rotation angle of the head position to determine a residual rotation angle, followed by dividing the residual rotation angle with the rotation angle of the head position. Examples of the second vestibulo ocular reflex data include data obtained by subtracting an angular velocity owing to CUS from an angular velocity of a head position during VOR, and the like. Namely, the second vestibulo ocular reflex data is data acquired by the indirect method.

Specifically, the second vestibulo ocular reflex data is preferably a value of VOR_{gain (I)} that is determined based on angular velocity data of the head of a subject and angular velocity data of eye movements of the subject, which are acquired by the angular velocity data-acquiring unit, according to the following equation. The angular velocity data-acquiring unit is set on the head of the subject and is configured to acquire the angular velocity data of the head and the angular velocity data of the eye movements when the head is turned.

VOR_{gain(I)}=(A−C)/A

In the above equation, A is an integral of the angular velocity data of the head and C is an integral of the angular velocity data of the eyes of the subject owing to catch-up saccades (CUS) during the time when the head is turned.

The first vestibulo ocular reflex data is data acquired by dividing a rotation angle owing to a vestibulo ocular reflex with a rotation angle of a head position. Examples of the first vestibulo ocular reflex data include a ratio between angles that are an integral of angular velocities of the head position and an integral of angular velocities of the eye position during VOR, respectively, an angular velocity ratio between the head position and eye position during a period from initiation of VOR to the predetermined time, and the like. Namely, the first vestibulo ocular reflex data is data acquired by the direct method.

Specifically, the first vestibulo ocular reflex data is preferably a value of the VOR_{gain (D)} determined based on the angular velocity data of the head and the angular velocity data of the eye movements acquired by the angular velocity data-acquiring unit according to the following equation.

VOR_{gain(D)}=B/A

In the above equation, A is the integral of the angular velocity data of the head, and B is an integral of the angular velocity data of the eye movements during the time when the head is turned.

### <Angular Velocity Data-Acquiring Process and Angular Velocity Data-Acquiring Unit>

The data acquiring process is a process of acquiring angular velocity data of the head of a subject and angular velocity data of eye movements of the subject when a data acquiring unit is set on the head of the subject and the head is turned. The data acquiring process is performed by the data acquiring unit.

The angular velocity data-acquiring unit includes a sensor configured to collect angular velocity data associated with rotational movements of the head of a subject, and a camera configured to capture eye movements of the subject. The angular velocity data-acquiring unit may further include other members as necessary.

The sensor is configured to detect movements (angular velocities) of the head when the head is turned. Examples of the sensor include acceleration sensors, gyroscopes, motion sensors (each composed of a gyroscope, an acceleration sensor, and a magnetic sensor), and the like. Use of the motion sensor enables measuring of a direction, angle, and velocity when the head of a subject is moved.

The camera is configured to detect eye movements of the subject when the head is turned. Examples of the camera include infrared CCD cameras, and the like. The infrared CCD camera is disposed at the right-eye side of the goggles, and is configured to capture images of pupils of the subject in real time. At maximum, 250 frames of images are captured per second to detect movements of eyeballs.

The sensor and the camera are mounted on the goggles that are configured to be set on the head of a subject.

The goggles are light in weight and have face cushions attached thereto, thus the goggles can be securely set on the head of a subject.

Examples of the above-mentioned other members include laser modules, and the like.

The laser module is mounted on goggles, and is capable of emitting laser light in 3 horizontal directions (left, center, right) to project the laser light onto a wall or screen, which is used as a calibration before a measurement and as a gaze target during a measurement.

### <Analysis Process and Analysis Unit>

The analysis process is a process that includes determining a value of VOR_{gain (I)} based on the angular velocity data of the head and the angular velocity data of the eye movements, which are acquired in the angular velocity data-acquiring process, according to the following equation, and analyzing semicircular canal functions based on the value of the VOR_{gain (I)}.

VOR_{gain(I)}=(A−C)/A

In the above equation, A is an integral of angular velocity data of the head, and C is an integral of angular velocity data of the eyes of the subject owing to catch-up saccades (CUS) during the time when the head is turned.

The data value of the A is an integral, and the data value of the C is a sum of a plurality of integrals.

Specifically, the data analysis unit is a laptop computer in which the analysis program is installed.

In the present invention, an indirect method is applied, not a direct method. The direct method is used for directly calculating an angular velocity of a head position and angular velocities of eye positions during VOR, and the indirect method is a method where angular velocities of CUS are subtracted from an angular velocity of a head position during VOR. Specifically, the direct method is a method in which VOR_{gain (D)} is calculated according to the following equation: VOR_{gain (D)}=B/A, where A is a data value of the angular velocity at the predetermined position on the head, and B is a data value of the angular velocity of the predetermined position of the eye of the subject. The indirect method is a method in which VOR_{gain (I)} is calculated according to the following equation: VOR_{gain (I)}=(A-C)/A, where A is a data value of the angular velocity of the predetermined position of the head, and C is a data value of catch-up saccades (CUS) at the predetermined position of the eye of the subject.

The data value of the A is an integral, and the data value of the C is a sum of a plurality of integrals.

According to the indirect method, VOR_{gain (I)} that is not influenced by artifacts induced by slippage and the like is obtained, thus accuracy of a test significantly improves.

A method of measuring VOR_{gain} in the vHIT will be explained with reference to figures hereinafter.

As a device specifically designed for vHIT, a vHIT device (ICS Impulse manufactured by Natus Medical Incorporated), which is a type where special goggles with a built-in high-speed camera and sensor are designed to be set on the head of a subject, is used to carry out a measurement.

The vHIT is performed in the following manner. First, a subject is asked to sit in a chair set approximately 1 m to 1.5 m away from a wall, and a target is set on the wall at the height with which the subject can view the target on the wall in front of the subject. The subject wears vHIT goggles. Next, a region of interest (ROI) is set. In the state where the subject gazes at the target that is set in the front of the subject, the position of the region of interest is adjusted so that a pupil comes at a center of the region of interest. Next, calibration for the horizontal direction is performed. The tester stands behind the subject, and securely holds the head or jaw of the subject with both hands. Next, the subject is instructed to gaze at the target, the head of the subject is rapidly turned at approximately 10 degrees in the horizontal direction, then the position of the head is held without turning back to the original position. The number of the times the rotational stimulus is applied varies depending on a type of a device used and is not particularly limited. Typically, the number of the times the rotational stimulus is applied is 20 times.

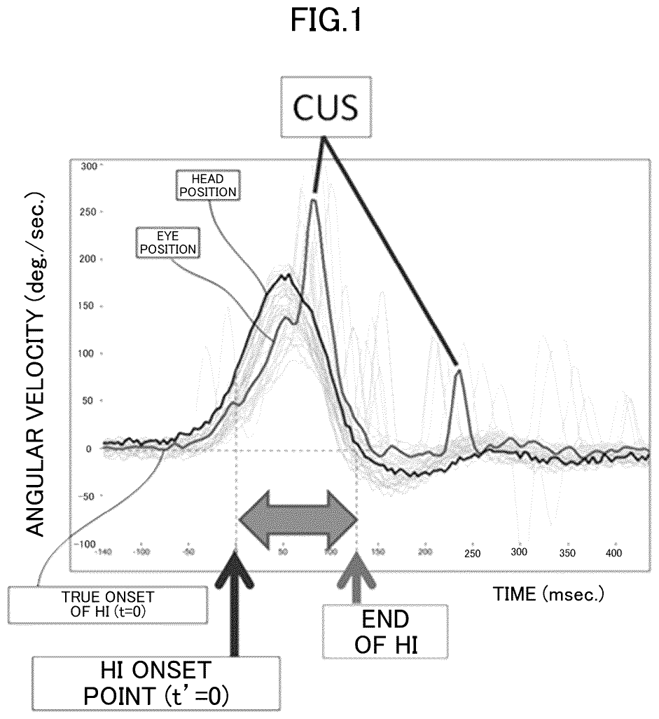

As the measurement of vHIT is performed using the vHIT device in the manner as described above, a graph depicting a relationship between a time and angular velocity is obtained as depicted in FIG. 1. In FIG. 1, the data for the 20 measurements is superimposed. Within the data for the 20 measurements, the results of the head position and the eye position indicated with thick lines are the results from one head impulse (HI). Two peaks associated with catch-up saccades (CUS) appear on the line of the eye position.

In FIG. 1, the true HI onset point (t=0) is an onset point of one HI, an HI onset point (t′=0) is an onset point on algorithm of one HI. The end of HI is an endpoint of one HI. Accordingly, the period from the HI onset (t′=0) to the end of HI is determined as a period when the head is turned with one HI.

Note that, in the data analysis performed by the vHIT device, an angular velocity is determined using the HI onset point (t′=0) as the standard, not the true HI onset point (t=0) in association with the calculation algorithm, but a similar result can be obtained even when the angular velocity is determined using the true HI onset point (t=0) as the standard.

Next, a method of determining VOR_{gain (D)} according to the direct method will be explained.

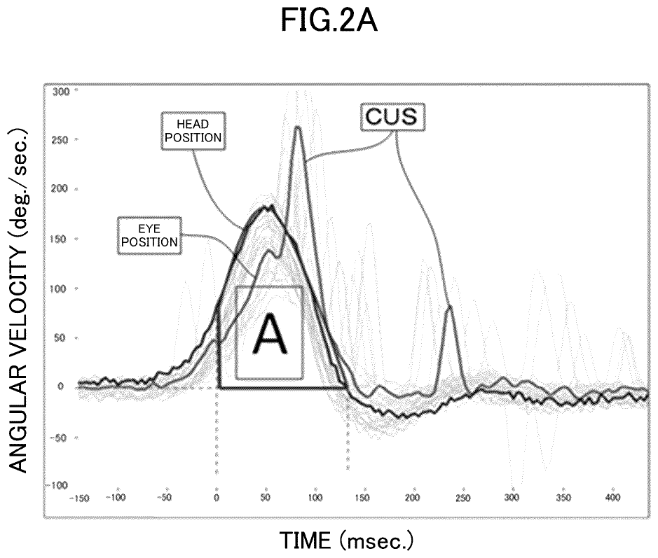

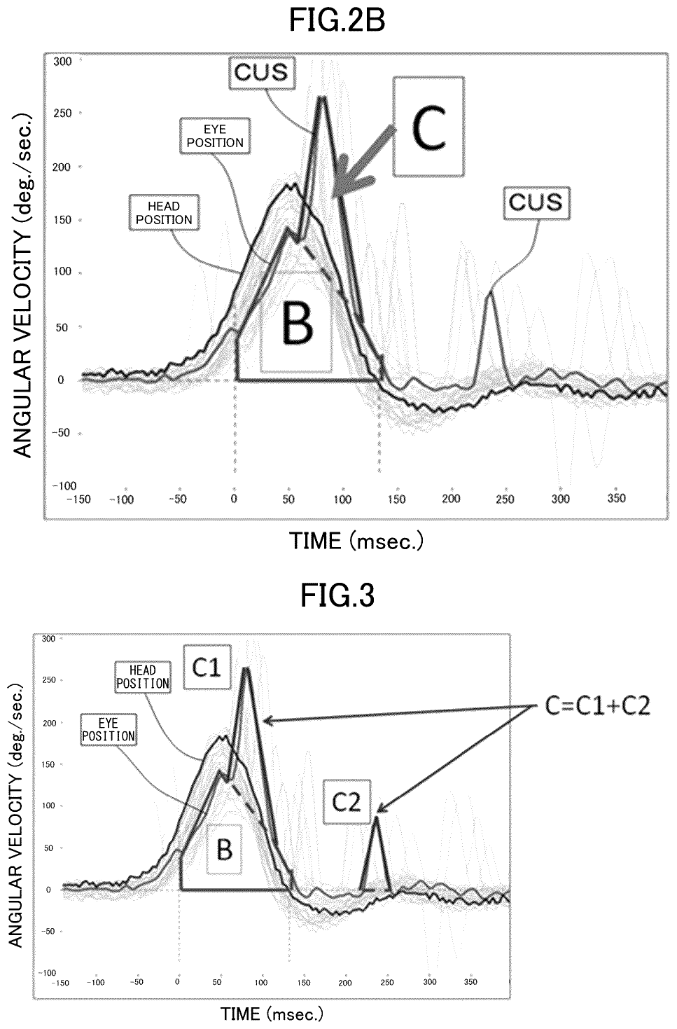

As depicted in FIGS. 2A and 2B, VOR_{gain (D)} is determined based on the data of the head position and eye movements of the subject acquired by the angular velocity data-acquiring unit, which is a vHIT device, when the head is turned, according to the following equation: VOR_{gain (D)}=B/A, where A is a data value of the angular velocity of the predetermined position of the head, and B is a data value of the angular velocity of the predetermined position of the eye of the subject when the head is turned.

For a calculation of the angular velocity of the head of VOR with one HI, as a rotation angle is an integral of angular velocities, the rotation angle of the head is the region defined by the thick lines in FIG. 2A, i.e., the region defined by a straight line where the angular velocity is 0 (bottom side), a straight line of the HI onset point (t′=0) (left side), VOR of the head position (top plane), and the HI end point (right side).

For calculation of the angular velocity B of the eye position of VOR with one HI, as a rotation angle is an integral of angular velocities, the rotation angle of the eye position is a region B defined by thick lines in FIG. 2B, i.e., a region defined by a straight line where the angular velocity is 0 (bottom side), a straight line of the HI onset point (t′=0) (left side), VOR of the eye position (top plane), and the HI end point (right side). If there is a change in the angular velocity of the eye position exceeding a certain level, such change is judged as CUS (=C) by the algorithm. CUS is not a change of the eye position owing to VOR, a rotation angle of the eye position is calculated by subtracting CUS appeared before ending of VOR. This calculation of subtraction of CUS is automatically carried out by the analysis program.

VOR_{gain (D)} is determined from the rotation angles A and B determined as described above, according to the following formula VOR_{gain (D)}=B/A, and an arithmetic mean of the values from the 20 measurements is determined as VOR_{gain (D)} of the direct method. The direct method may be referred to as a direct method (angle ratio) hereinafter.

VOR_{gain (D)} may be also directly determined from a ratio between the angular velocity of the eye position and the angular velocity of the head position during a period from the VOR onset point and the predetermined time (e.g., 60 msec, 100 msec). Such direct method may be referred to as a direct method (angular velocity ratio at 60 ms) or a direct method (angular velocity ratio at 100 ms) hereinafter.

Next, a method of determining VOR_{gain (I)} according to the indirect method will be explained.

According to the theory of the indirect method, the following hypothesis may be established. That is, as long as a subject continues to gaze at a target, a rotation angle (an integral of angular velocities) of a head position and a rotation angle (an integral of angular velocities) of an eye position become identical after a certain period of time regardless of presence or absence of impairments in the semicircular canal functions.

If the above-described hypothesis is correct, as depicted in FIG. 3, a relationship of a rotation angle A=B+C is satisfied, thus VOR_{gain (I)} can be determined according to the following equation: VOR_{gain (I)}=(A-C)/A, based on the data of the head movements and eye movements of the subject acquired by the data acquiring unit that is a vHIT device, when the head is turned. In the above equation, A is a data value of the angular velocity of the predetermined position of the head, and C is a data value of catch-up saccades (CUS) at the predetermined position of the eye of the subject, when the head is turned.

The data value of the A is preferably an integral, and the data value of the C is preferably a sum of a plurality of integrals of the data values.

The data value of the A is a rotation angle (an integral of the angular velocities) of the head position with head impulse (HI).

The data value of the C is an area (an integral of the angular velocities) of multiple CUS. The catch-up saccade (CUS) appears a few times before and after the end of VOR, thus a total area of CUS is determined as C.

Accordingly, the present inventor has found a calculation method (indirect method) where the sum of the integrals of CUS is subtracted from the integral of the head position of VOR without using the integral B of the eye position during one HI. The catch-up saccade (CUS) appears a few times before and after the end of VOR, thus a total area of CUS is determined as C.

The analysis unit is configured to acquire a plurality of values of the VOR_{gain (I)}, determine a line of best fit based on the acquired values of the VOR_{gain (I)}, and determine a value of the VOR_{gain (I)} at a certain angular velocity based on the line of best fit. Note that, the line of best fit includes a straight line of best fit and a curve of best fit.

The angular velocity in the rotational stimulation test is preferably from 100°/sec to 200°/sec. Accordingly, for example, VOR_{gain} at the angular velocity of 150°/sec can be determined from line of best fit, and VOR_{gain (I)} that is not affected by the angular velocity can be obtained. Therefore, the VOR_{gain} values can be standardized.

As a method of determining the line of best fit, various statistical analysis methods can be used. Examples of the method include the least squares method and the like.

The analysis unit is configured to acquire a plurality of values of the VOR_{gain (I)}, determine a line of best fit based on the acquired values of the VOR_{gain (I)}, and determine dispersion of the acquired values of the VOR_{gain (I)} based on the line of best fit. As a result, accuracy of tests can be improved.

As vHIT is performed several times, a scatter diagram of the angular velocities and VOR_{gain} is obtained. A line of best fit is determined from the scatter diagram so that VOR_{gain} at a certain angular velocity is calculated.

As a method of determining the line of best fit, various statistical analysis methods can be used. Examples of the method include the least squares method and the like.

Examples of the dispersion include a standard deviation, a standard error, a variance, a coefficient of variation (CV), and the like.

The analysis unit is capable of determining and analyzing a standard deviation of the differences each between the VOR_{gain (D)} determined by the direct method and the VOR_{gain (I)} determined by the indirect method to find out the degree of artifacts. Therefore, whether a test is carried out under appropriate conditions or not can be evaluated.

Moreover, a scatter diagram is drawn from the VOR_{gain (D)} of several subjects determined by the direct method and the VOR_{gain (I)} of the several subjects determined by the indirect method and a correlation coefficient is determined so that the data of the patients can be analyzed precisely.

### <Other Processes and Other Units>

Other processes are not particularly limited, and may be appropriately selected according to the intended purpose. Examples of other processes include a communication process, an input process, and the like.

Other units are not particularly limited, and may be appropriately selected according to the intended purpose. Examples of other units include communication unit, an input unit, and the like.

The communication unit is not particularly limited, provided that the communication unit can communicate with a plurality of testing sites or the like. Any unit known in the related art may be appropriately used as the communication unit. Examples of the communication unit include transceivers, information communication networks, the internet, and the like.

The input unit is not particularly limited, provided that the input unit can receive various demands for the first and second analysis systems of the present invention. Any unit known in the related art may be appropriately used as the input unit. Examples of the input unit include keyboards, mouses, touch panels, microphones, and the like.

### (Analytical Data Acquisition Device)

The analytical data acquisition device of the present invention is used for analysis of semicircular canal functions with rotational stimuli, and includes a sensor configured to collect angular velocity data associated with rotational movements of the head of a subject, and a camera configured to capture eye movements of the subject. The analytical data acquisition device may further include other units as necessary.

The analytical data acquisition device is not particularly limited, and may be appropriately selected according to the intended purpose. The analytical data acquisition device is preferably a goggles-type analytical data acquisition device.

The goggles-type analytical data acquisition device includes a high-speed camera capable of capturing 250 images per second at the maximum as high-speed photography, and a sensor for acquiring information of a head position.

Examples of the above-mentioned other units include analytical software, personal computers, and the like.

Since the analytical data acquisition device is very portable, a test can be carried out at the bed side of a patient or during consultation of an outpatient.

### (Analysis Program)

In the first embodiment, the analysis program of the present invention is an analysis program for analysis of semicircular canal functions with rotational stimuli, and the analysis program causes a computer to execute processes of: acquiring first vestibulo ocular reflex data and second vestibulo ocular reflex data, where the first vestibulo ocular reflex data is acquired by dividing a rotation angle owing to a vestibulo ocular reflex with a rotation angle of a head position, and the second vestibulo ocular reflex data is acquired by subtracting at least a rotation angle owing to catch-up saccades (CUS) from the rotation angle of the head position to determine a residual rotation angle, followed by dividing the residual rotation angle with the rotation angle of the head position; and analyzing semicircular canal functions based on the second vestibulo ocular reflex data, or based on both the first vestibulo ocular reflex data and the second vestibulo ocular reflex data.

In the second embodiment, the analysis program of the present invention is an analysis program for analysis of semicircular canal functions with rotational stimuli, and the analysis program causes a computer to execute processes of: acquiring angular velocity data of the head of a subject and angular velocity data of eye movements of the subject when the head of the subject is turned; determining VOR_{gain (I)} based on the angular velocity data of the head and the angular velocity data of the eye movements according to the following equation: VOR_{gain (I)}=(A-C)/A, where A is an integral of the angular velocity data of the head and C is an integral of the angular velocity data of the eyes of the subject owing to catch-up saccades (CUS) during the time when the head is turned; and analyzing semicircular canal functions based on the value of VOR_{gain (I)}.

The analysis programs of the first and second embodiments of the present invention may be, for example, programs that cause a computer to execute the analysis methods of the first and second embodiments of the present invention. Moreover, preferable embodiments of the analysis programs of the first and second embodiments of the present invention are, for example, identical to the preferable embodiments of the analysis method of the first and second embodiments of the present invention.

The analysis programs of the first and second embodiments of the present invention can be created in various program languages known in the related art depending on configurations of a computer system for use and a type or version of an operating system.

The analysis program of the first and second embodiments of the present invention may be written on recording media, such as internal hard disk drives, external hard disk drives, and the like, or recording media such as CD-ROMs, DVD-ROMs, MO disks, USB memory devices, and the like.

When the analysis program of the first and second embodiments of the present invention is written on the above-mentioned recording medium, moreover, the analysis program is used directly via a recording media reader included in a computer system, or used by installing on a hard disk from the recording media reader, as necessary. Moreover, the analysis programs of the first and second embodiments of the present invention may be recorded on an external memory region (e.g., another computer and the like) that is accessible from the computer system via the information communication network. In this case, the analysis programs of the first and second embodiments of the present invention recorded in the external memory region can be used directly from the external memory region via the information communication network, or can be used by installing on a hard disk from the external memory region via the information communication network, as necessary.

The analysis programs of the first and second embodiments of the present invention may be each recorded across multiple recording media by dividing the analysis program into the predetermined processes.

### <Computer-Readable Recording Medium>

The computer-readable recording medium associated with the present invention stores the analysis program of the present invention.

The computer-readable recording medium associated with the present invention is not particularly limited, and may be appropriately selected according to the intended purpose. Examples of the computer-readable recording medium include internal hard disk drives, external hard disk drives, CD-ROMs, DVD-ROMs, MO disks, USB memory devices, and the like.

Moreover, the computer-readable recording medium associated with the present invention may be multiple recording media across which the analysis program of the present invention is divided into arbitrary processes, and stored.

One example of the technology disclosed in the present invention will be specifically explained with reference to configuration examples of the device, a flowchart, and the like, hereinafter.

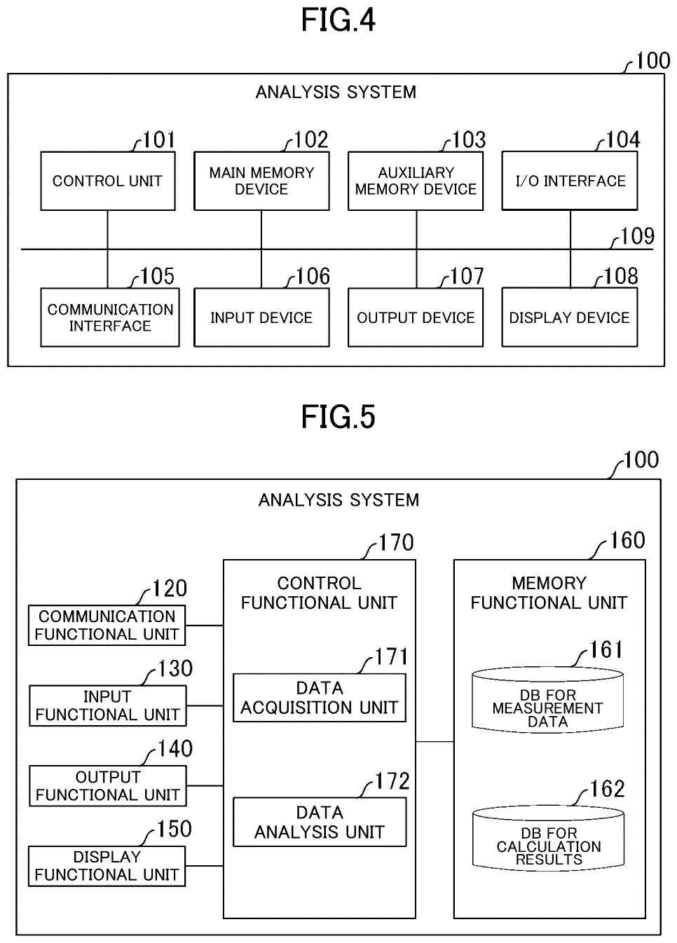

FIG. 4 illustrates a hardware configuration example of the analysis system of the present invention.

In the analysis system 100 of the present invention depicted in FIG. 4, for example, a control unit 101, a main memory device 102, an auxiliary memory device 103, an I/O interface 104, a communication interface 105, an input device 106, an output device 107, and a display device 108 are connected via a system bus 109.

The control unit 101 is configured to control operations (e.g., four arithmetic operations, comparison operations, and the like), control operations of hardware and software, and the like. For example, the control unit 101 may be a central processing unit (CPU), or a part of a machine used for the analysis system of the present invention, or a combination thereof.

For example, the control unit 101 executes a program (e.g., the analysis program of the present invention) read by the main memory device 102 to perform various functions.

For example, operations performed by the control functional unit of the analysis system of the present invention can be performed by the control unit 101.

As well as storing various programs, the main memory device 102 stores data, etc., necessary to execute the various programs. As the main memory device 102, for example, a memory device including at least either a read only memory (ROM) or random-access memory (RAM) can be used.

The ROM stores various programs, such as basic input/output systems (BIOS) and the like. The ROM is not particularly limited, and may be appropriately selected according to the intended purpose. Examples of the ROM include Mask ROMs, programmable ROMs (PROMs), and the like.

The RAM functions, for example, as a working area into which various programs recorded on ROM, the auxiliary memory device 103, or the like, are expanded, when the programs are executed by the control unit 101. The RAM is not particularly limited, and may be appropriately selected according to the intended purpose. Examples of the RAM include dynamic random-access memories (DRAMs), static random-access memories (SRAMs), and the like.

The auxiliary memory device 103 is not particularly limited, provided that the auxiliary memory device 103 can store various information. The auxiliary memory device 13 may be appropriately selected according to the intended purpose. Examples of the auxiliary memory device 103 include solid state drives (SSD), hard disk drives (HDD), and the like. Moreover, the auxiliary memory device 103 may be a removable memory drive, such as a CD drive, a DVD drive, a Blu-ray® Disc (BD) drive, and the like.

For example, moreover, the analysis program of the present invention is stored in the auxiliary memory device 103, is loaded onto a RAM (main memory) of the main memory device 102, and then is executed by the control unit 101.

The I/O interface 104 is an interface for connecting to various external devices. The I/O interface 104 enables input and output of data of compact disc ROMs (CD-ROMs), digital versatile disk ROMs (DVD-ROMs), Magneto-Optical disks (MO disks), USB memory devices [Universal Serial Bus (USB) flash drives], or the like.

The communication interface 105 is not particularly limited, and any communication interface known in the related art may be suitably used. Examples of the communication interface 105 include a wireless or wired communication device, and the like.

The input device 106 is not particularly limited, provided that the input device 106 can receive various demands for the analysis system 100 of the present invention or input of information, and any input device known in the related art may be suitably used. Examples of the input device 106 include keyboards, mouses, touch panels, microphones, and the like. In the case where the input device 106 is a touch panel (touch panel display), moreover, the input device 106 can also function as a display device 108.

The output device 107 is not particularly limited, and any output device known in the related art may be suitably used. Examples of the output device 107 include printers and the like.

The display device 108 is not particularly limited, and any display device known in the related art may be suitably used. Examples of the display device 108 include liquid crystal displays, organic EL display, and the like.

FIG. 5 illustrates a functional configuration example of the analysis system of the present invention.

As depicted in FIG. 5, the analysis system 100 of the present invention includes a communication functional unit 120, an input functional unit 130, an output functional unit 140, a display functional unit 150, a memory functional unit 160, and a control functional unit 170.

For example, the communication functional unit 120 receives and transmits various data from and to an external device. For example, the communication functional unit 120 may receive data, such as data of head movements and eye movements of a subject, from an external device.

For example, the input functional unit 130 receives various instructions for the analysis system 100 of the present invention. For example, moreover, the input functional unit 130 receives information, such as attributes of a subject and the like.

For example, the output functional unit 140 prints out a measurement result, such as VOR_{gain} of the direct method and the indirect method, a standard deviation of differences between VOR_{gain} of the direct method and VOR_{gain} of the indirect method, and the like.

For example, the display functional unit 150 displays a measurement result, such as VOR_{gain} of the direct method and the indirect method, a standard deviation of differences between VOR_{gain} of the direct method and VOR_{gain} of the indirect method, and the like, on a display panel.

For example, the memory functional unit 160 stores DB for measurement data 161 and DB for calculation results 162, as well as storing various programs.

The control functional unit 170 includes a data acquisition unit 171 and a data analysis unit 172. For example, the control functional unit 170 is configured to control the operation of the entire analysis system 100 of the present invention, as well as executing various programs stored in the memory functional unit 160.

For example, the data acquisition unit 171 is configured to acquire data of the head positions and eye movements of the subject when the head of the subject is turned.

For example, the data analysis unit 172 carries out a process of analyzing the following equation: VOR_{gain (I)}=(A-C)/A, based on the data, where A is a data value of a rotation angle of the predetermined position of the head, and C is a data value of catch-up saccades (CUS) at the predetermined position of the eye of the subject, when the head is turned.

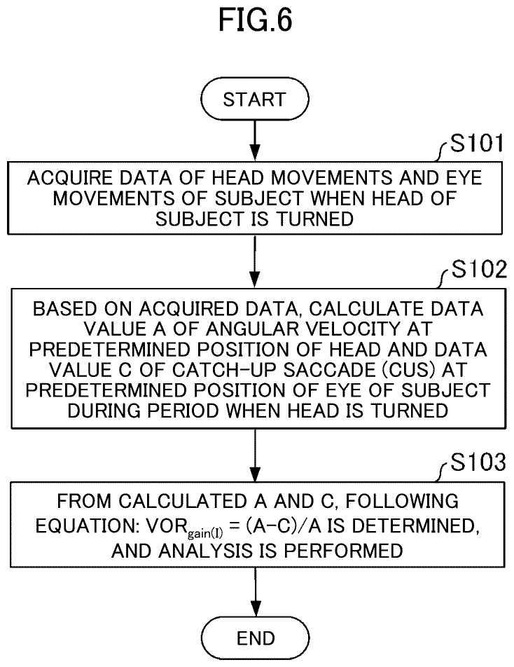

FIG. 6 is a flowchart illustrating one example of a few of the processes in the analysis method of the present invention. The flow of the processes of the analysis method of the present invention will be explained with reference to FIG. 5 hereinafter.

In the step S101, once the data acquisition unit 171 in the control functional unit 170 of the analysis system 100 acquires data of the head movements and eye movements of the subject when the head of the subject is turned, the processing is moved onto S102.

In the step S102, once the data analysis unit 172 in the control functional unit 170 of the analysis system 100 calculates a data value A of a rotation angle of the predetermined position of the head, and a data value C of the catch-up saccade (CUS) at the predetermined position of the eye of the subject during the time when the head is turned, based on the data, the processing is moved onto S103.

In the step S103, once the data analysis unit 172 in the control functional unit 170 of the analysis system 100 determines VOR_{gain (I)} from the data values A and C according to the following equation: VOR_{gain (I)}=(A-C)/A, the processing is completed.

According to the analysis method and analysis system of the present invention using the analysis program of the present invention, results are not affected by artifacts induced by slippage and the like, accuracy of tests is improved, and analysis of semicircular canal functions can be performed accurately and precisely.

### EXAMPLES

Examples of the present invention will be explained hereinafter, but the present invention is not limited to these Examples in any way.

In Examples below, <Equation of linear regression on scatter diagram of peak angular velocity and VOR_{gain}>, <Definition of each item and each equation>, and <Standard deviation and standard error> in definition equations of linear regression are as follows.

<Equation of Linear Regression on Scatter Diagram of Peak Angular Velocity and VOR_{gain}>

An equation of linear regression of the gains, which are dependent to peak angular velocity, according to estimated regression analysis from samples is determined as:

$$y = \frac{{S}_{xy}}{{S}_{xx}} ( x - \overline{x} ) + \overline{y}$$

and a correlation coefficient is determined as:

$$r = \frac{{S}_{xy}}{\sqrt{{S}_{xx} {S}_{yy}}} .$$

In the above equations, Sₓₓ, S_{yy}, S_{xy}, x̅ (x-bar), and y̅ (y-bar) are a variance of the peak angular velocity, a variance of the gain, the mean peak angular velocity, and the mean gain, respectively.

The coefficients a and b of the straight-line equation: y=ax+b, and the correlation coefficient r are defined by the following equations.

### Definition of Each Item and Each Equation

xᵢ: a peak angular velocity of the i-th sample (“i” is a number in the order)
yᵢ: a gain of the i-th sample
n: the number of samples
x̅(x-bar): the mean of peak angular velocities of all the samples

$$\overline{x} = \frac{1}{n} {\sum }_{k = 1}^{n} {x}_{k} \qquad ( 1 )$$

y̅: the mean of gains of all the samples

$$\overline{y} = \frac{1}{n} {\sum }_{k = 1}^{n} {y}_{k} \qquad ( 2 )$$

Sxx: a variance associated with a x component

$$\begin{matrix}{S}_{xx} & = & \frac{1}{n} {\sum }_{k = 1}^{n} {( {x}_{k} - \overline{x} )}^{2} & ( 3 ) \\ & = & \frac{1}{n} ( {\sum }_{k = 1}^{n} {x}_{k}^{2} - \frac{1}{n} {( {\sum }_{k = 1}^{n} {x}_{k} )}^{2} ) & ( 4 )\end{matrix}$$

Since the calculation of a variance by calculating the mean x̅ (x-bar) of xₖ of all the samples, followed by calculating xₖ-x̅ (x-bar) for each Xₖ causes duplication of the effort, in reality, calculation of the mean and calculation of the variance are carried out at once by modifying the calculation of the equation (3) to calculate the equation (4) with the implemented software. Similarly, the second line of each definition equation is the equation used for the actual calculation, hereinafter.
Syy: a variance associated with the y component

$$\begin{matrix}{S}_{yy} & = & \frac{1}{n} {\sum }_{k = 1}^{n} {( {y}_{k} - \overline{y} )}^{2} & ( 5 ) \\ & = & \frac{1}{n} ( {\sum }_{k = 1}^{n} {y}_{k}^{2} - \frac{1}{n} {( {\sum }_{k = 1}^{n} {y}_{k} )}^{2} ) & ( 6 )\end{matrix}$$

Sxy: covariance associated with the x component and y component

$$\begin{matrix}{S}_{xy} & = & \frac{1}{n} {\sum }_{k = 1}^{n} ( {x}_{k} - \overline{x} ) ( {y}_{k} - \overline{y} ) & ( 7 ) \\ & = & \frac{1}{n} ( {\sum }_{k = 1}^{n} {x}_{k} {y}_{k} - \frac{1}{n} ( {\sum }_{k = 1}^{n} {x}_{k} ) ( {\sum }_{k = 1}^{n} {y}_{k} ) ) & ( 8 )\end{matrix}$$

a and b: coefficients of linear regression (regression coefficients, a: slope, and b: y-intercept)

$$\begin{matrix}a & = & \frac{{S}_{xy}}{{S}_{xx}} & ( 9 ) \\ & = & \frac{n {\sum }_{k = 1}^{n} {x}_{k} {y}_{k} - {\sum }_{k = 1}^{n} {x}_{k} {\sum }_{k = 1}^{n} {y}_{k}}{n {\sum }_{k = 1}^{n} {x}_{k}^{2} - {( {\sum }_{k = 1}^{n} {x}_{k} )}^{2}} & ( 10 )\end{matrix}$$

$$\begin{matrix}b & = & \overline{y} - \frac{{S}_{xy}}{{S}_{xx}} \overline{x} = \frac{{S}_{xx} \overline{y} - {S}_{xy} \overline{x}}{{S}_{xx}} & ( 11 ) \\ & = & \frac{{\sum }_{k = 1}^{n} {x}_{k}^{2} {\sum }_{k = 1}^{n} {y}_{k} - {\sum }_{k = 1}^{n} {x}_{k} {\sum }_{k = 1}^{n} {x}_{k} {y}_{k}}{n {\sum }_{k = 1}^{n} {x}_{k}^{2} - {( {\sum }_{k = 1}^{n} {x}_{k} )}^{2}} & ( 12 )\end{matrix}$$

r: a correlation coefficient

$$\begin{matrix}r & = & \frac{{S}_{xy}}{\sqrt{{S}_{xx} {S}_{yy}}} & ( 13 ) \\ & = & \frac{n {\sum }_{k = 1}^{n} {x}_{k} {y}_{k} - {\sum }_{k = 1}^{n} {x}_{k} {\sum }_{k = 1}^{n} {y}_{k}}{\sqrt{( n {\sum }_{k = 1}^{n} {x}_{k}^{2} - {( {\sum }_{k = 1}^{n} {x}_{k} )}^{2} ) ( n {\sum }_{k = 1}^{n} {y}_{k}^{2} - {( {\sum }_{k = 1}^{n} {y}_{k} )}^{2} )}} & ( 14 )\end{matrix}$$

r²: a coefficient of determination

### Standard Deviation and Standard Error

σₓ: a standard error of the peak angular velocity (a square of the unbiased variance), where the unbiased variance means an unbiased estimator of a population variance.

$${σ}_{x} = \sqrt{\frac{1}{n - 1} {\sum }_{k = 1}^{n} {( {x}_{k} - \overline{x} )}^{2}} \qquad ( 15 )$$

σ_{y}: a standard error of a gain (a square of the unbiased variance)

$${σ}_{y} = \sqrt{\frac{1}{n - 1} {\sum }_{k = 1}^{n} {( {y}_{k} - \overline{y} )}^{2}} \qquad ( 16 )$$

SE_{R}: a standard error of regression/residual standard error

$$\begin{matrix}{SE}_{R} & = & \sqrt{\frac{1}{n - 2} {\sum }_{k = 1}^{n} {e}_{k}^{2}} = \sqrt{\frac{1}{n - 2} {\sum }_{k = 1}^{n} {( {y}_{k} - ( {ax}_{k} + b ) )}^{2}} & \\ & = & \sqrt{\frac{{\sum }_{k = 1}^{n} {( {y}_{k} - \overline{y} )}^{2} - a {\sum }_{k = 1}^{n} {( {x}_{k} - \overline{x} )}^{2}}{n - 2}} & ( 17 ) \\ & = & \sqrt{\frac{\begin{matrix}( n {\sum }_{k = 1}^{n} {y}_{k}^{2} - {( {\sum }_{k = 1}^{n} {y}_{k} )}^{2} ) - \\ a ( n {\sum }_{k = 1}^{n} {x}_{k} {y}_{k} - {\sum }_{k = 1}^{n} {x}_{k} {\sum }_{k = 1}^{n} {y}_{k} )\end{matrix}}{n ( n - 2 )}} & ( 18 )\end{matrix}$$

SE_{RC}: the correction equation of performing calculation by replacing a sum of squares of the arithmetic mean of the residuals (eᵢ) of the linear regression with a sum of squares of the arithmetic mean of distances (e′ᵢ) from the line of the linear regression.

$${e}_{i}^{′} = \frac{1}{√ ( 1 + {a}^{2} )} {e}_{i} \qquad ( 19 )$$

$${SE}_{RC} = \sqrt{\frac{1}{n - 2} {\sum }_{k = 1}^{n} {e}_{k}^{′2} = \frac{1}{√ ( 1 + {a}^{2} )} {SE}_{R}} \qquad ( 20 )$$

### Example 1

In the direct method and the indirect method, whether presence or absence of slippage-induced artifacts affected a value of VOR_{gain} of a patient having unilateral impairments of semicircular canal functions was determined.

As a device specifically designed for vHIT, a vHIT device (ISC Impulse manufactured by Natus Medical Incorporated) was used to measure VOR_{gain} according to the direct method (angle ratio), direct method (angular velocity ratio at 60 ms), and indirect method. The used vHIT device was a type of device where special goggles, in which the analysis program of the present invention was installed, and a high-speed camera and a sensor were built-in, were designed to be set on the head of a subject. A VOR_{gain} value of the direct method (angular velocity ratio at 60 ms) is a value directly determined from a ratio between an angular velocity of an eye position and an angular velocity of a head position during a period from a VOR onset point to 60 msec. The ISC Impulse (manufactured by Natus Medical Incorporated) in which the analysis program of the present invention is installed can measure VOR_{gain} according to both the direct method and the indirect method.

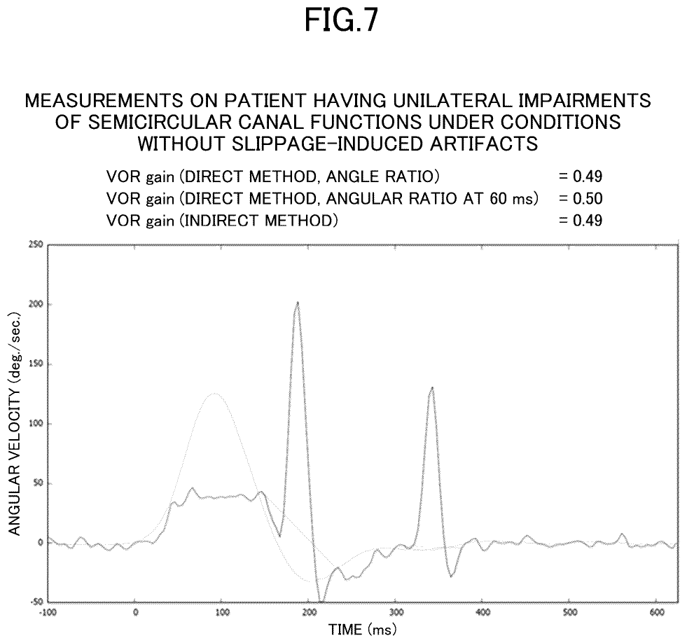

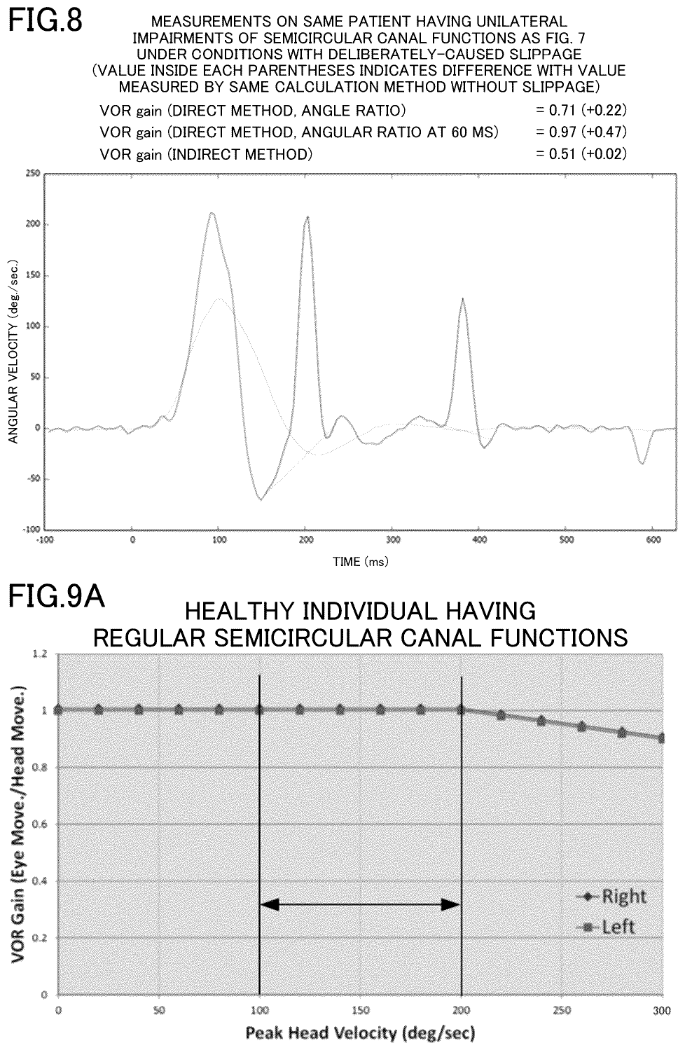

As depicted in FIG. 7, in the conditions without slippage-induced artifacts, VOR_{gain} of the direct method (angle ratio) was 0.49, VOR_{gain} of the direct method (angular velocity ratio at 60 ms) was 0.50, VOR_{gain} of the indirect method was 0.49, there was no noticeable difference in the results between the direct method and the indirect method.

Slippage-induced artifacts were intentionally caused for a patient having unilateral impairments of semicircular canal functions as in FIG. 7, and VOR_{gain} was measured according to the direct method and the indirect method in the same manner as described above. Specifically, head impulse was applied in the state where the hands of the tester touched the goggles so that the impulse was directly transmitted to the goggles to cause significant slippage-induced artifacts. In order to perform an appropriate test without slippage-induced artifacts, it is necessary to perform a test with holding positions as far as possible from the goggles. In this case, the test was performed with holding the band of the goggles when the tester held the head of the subject to intentionally induce slippage-induced artifacts. The results are depicted in FIG. 8.

According to the results depicted in FIG. 8, VOR_{gain} of the direct method (angular ratio) was 0.71, VOR_{gain} of the direct method (angular velocity ratio at 60 ms) was 0.97, and VOR_{gain} of the indirect method was 0.51.

Because of the presence of the slippage-induced artifacts, VOR_{gain} of the direct method (angular ratio) was increased by 0.22, from 0.49 to 0.71, and VOR_{gain} of the direct method (angular velocity ratio at 60 ms) was increased by 0.47, from 0.50 to 0.97, whereas VOR_{gain} of the indirect method was increased only by 0.02, from 0.49 to 0.51 due to the slippage-induced artifacts.

It was found from the results presented above that the result of the indirect method was not affected by the slippage-induced artifacts in comparison with the direct method. Therefore, it was found that an accurate semicircular canal function test could be performed according to the indirect method, and accuracy of the test was significantly improved compared to the indirect method.

### Example 2

A relationship between an angular velocity and a VOR_{gain} value when a rotational stimulus was applied was investigated for a healthy individual having regular semicircular canal functions and a patient having impaired semicircular canal functions.

Like Example 1, a relationship between an angular velocity and a value of VOR_{gain (I)} when a rotational stimulus was applied was determined according to the indirect method using ICS Impulse (manufactured by Natus Medical Incorporated), in which the analysis program of the present invention was installed.

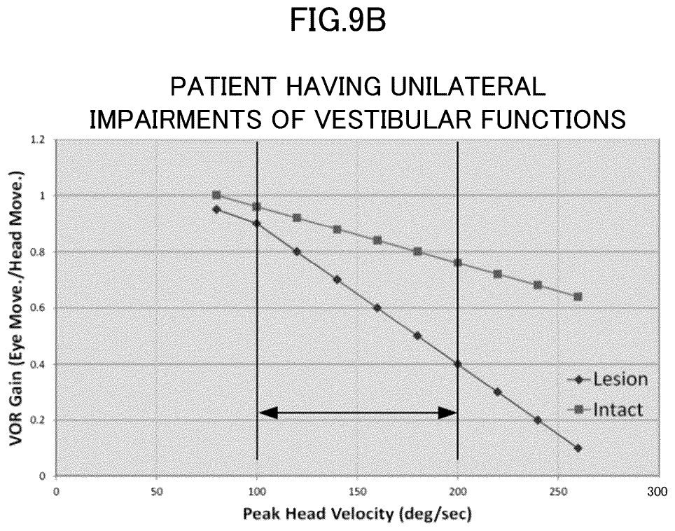

As depicted in FIG. 9A, the healthy individual having regular semicircular canal functions demonstrated an unnoticeable change even when an angular velocity was changed, and the value of VOR_{gain (I)} was approximately 1.0 and was constant in the angular velocity range of 100°/sec to 200°/sec.

Meanwhile, as depicted in FIG. 9B, the patient having unilateral impairments of vestibular function demonstrated a decline in the value of VOR_{gain (I)} as the angular velocity increased when a rotational stimulus was applied, the value of VOR_{gain (I)} at the angular velocity of 100°/sec was 0.9, and the value of VOR_{gain (I)} at the angular velocity of 200°/sec was reduced to 0.4. As described above, it is possible to intentionally vary a value of VOR_{gain (I)} of the patient having unilateral impairments of vestibular function depending on the angular velocity when a rotational stimulus is applied, thus an accurate test may not be performed.

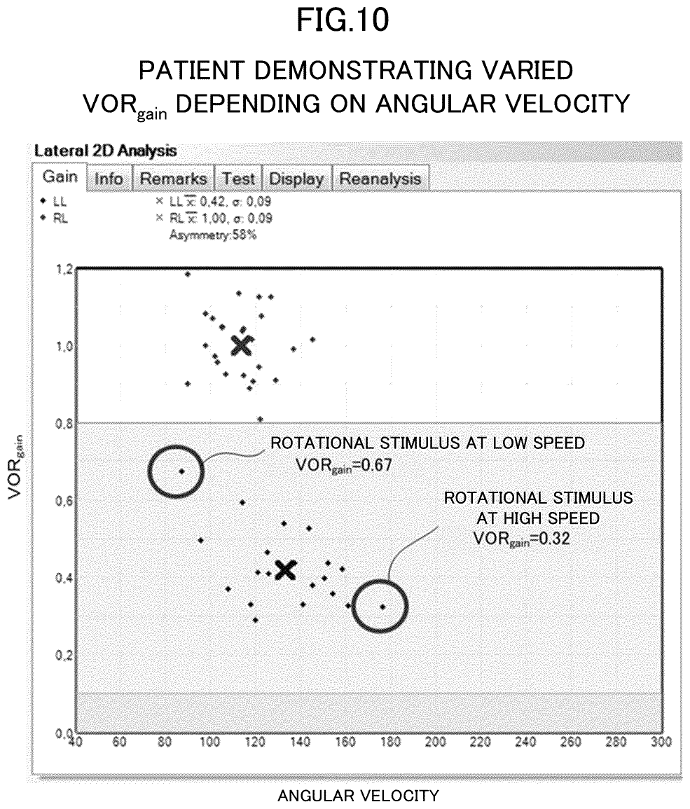

Moreover, FIG. 10 depicts an example where VOR_{gain} of a patient having impaired left-side vestibular function varied depending on the change of the angular velocity when a rotational stimulus was applied.

In the bottom part of the graph of FIG. 10 depicting the results of the left eye, VOR_{gain} was 0.67 when a rotational stimulus was applied at the low-speed angular velocity of 90°/sec, and VOR_{gain} was 0.32 when a rotational stimulus was applied at the high-speed angular velocity of 175°/sec. It was found from the above-mentioned results that the difference due to the angular velocity was 0.35, which was a significant. Note that, “x” in FIG. 10 indicates the mean value.

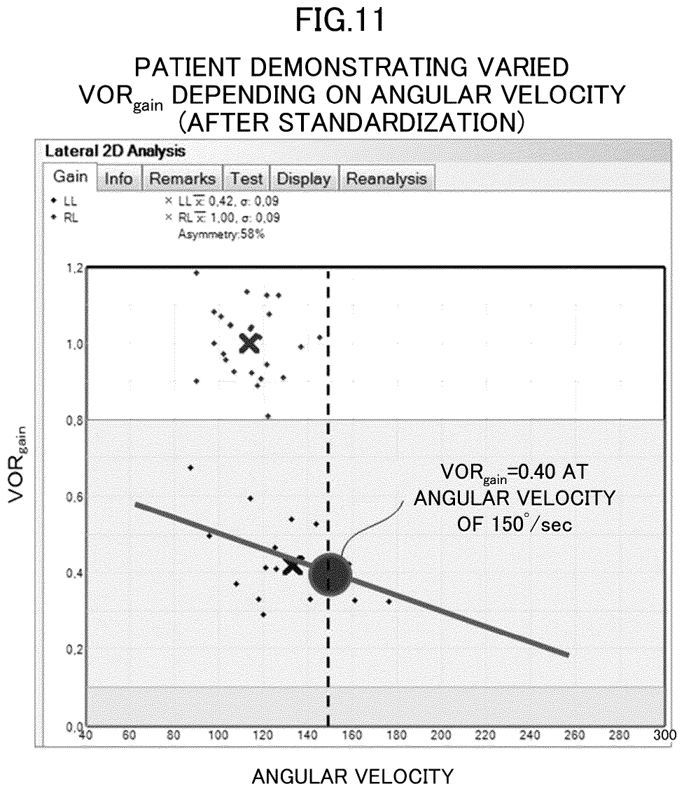

Therefore, a plurality of values of VOR_{gain (I)} (8 or more values at different angular velocities) was acquired as depicted in FIG. 11, a line of best fit of the acquired values of the VOR_{gain (I)} was determined according to the least squares method, and a value of VOR_{gain} at a certain angular velocity was determined based on the line of best fit. In FIG. 11, for example, VOR_{gain} was 0.40 at the angular velocity of 150°/sec. Note that, the line of best fit includes a straight line of best fit and a curve of best fit.

Accordingly, for example, VOR_{gain} at the angular velocity of 150°/sec can be determined from the line of best fit, VOR_{gain (I)} that is not significantly affected by the angular velocity can be obtained, and the VOR_{gain} value can be standardized.

Note that, the line of best fit according to the least squares method can be determined by the equations (10), (12), and (14), presented above.

### Example 3

A standard deviation (a) was measured as an item for evaluation of test accuracy by measuring VOR_{gain} 20 times each for left and right by ICS Impulse (manufactured by Natus Medical Incorporated), in which the analysis program of the present invention was installed. Note that, the standard deviation (a) is a value determined by the above-presented equation (16).

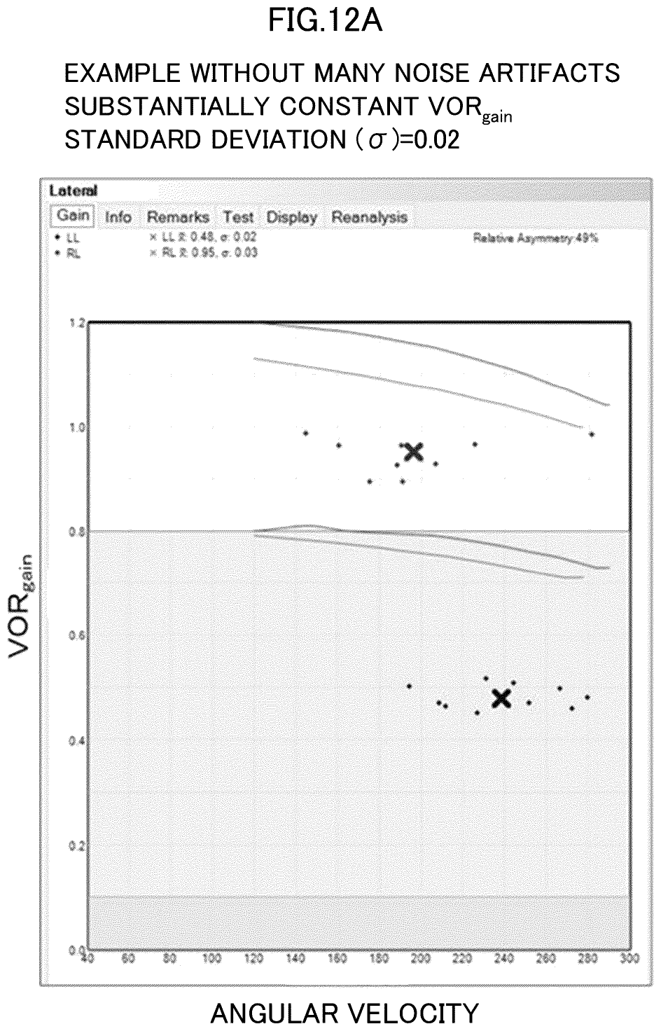

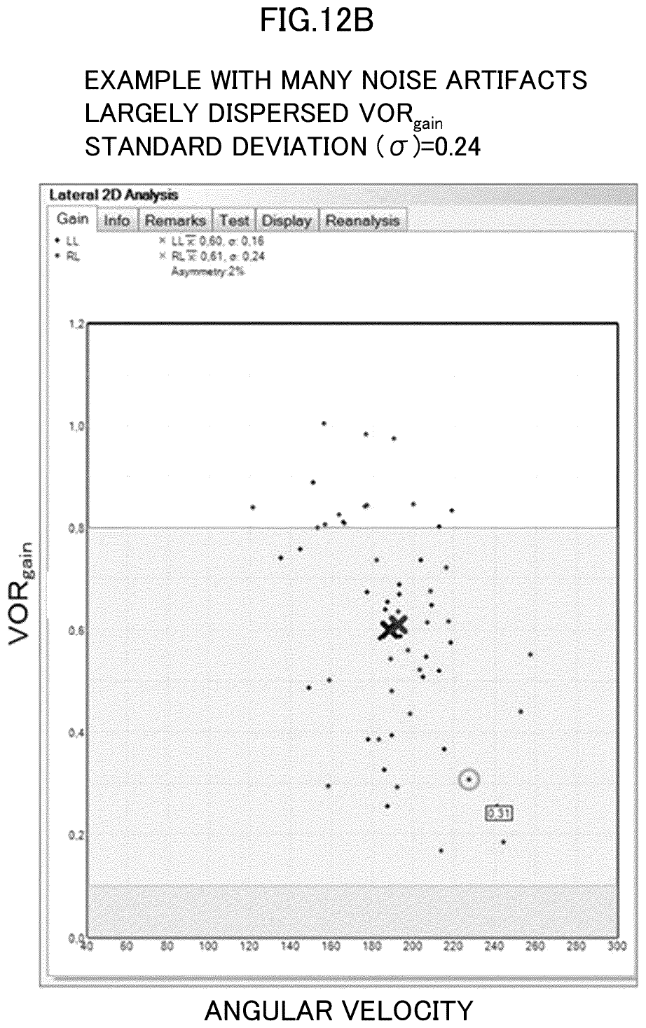

FIG. 12A depicts an example without many noise artifacts, where VOR_{gain} is substantially constant and the standard deviation (a) is 0.02. On the other hand, FIG. 12B depicts an example with many noise artifacts, where VOR_{gain} varies, and the standard deviation (a) is 0.24.

As depicted in FIGS. 12A and 12B, test accuracy can be evaluated with a standard deviation (a) measured by applying rotational stimulus 20 times each for left and right.

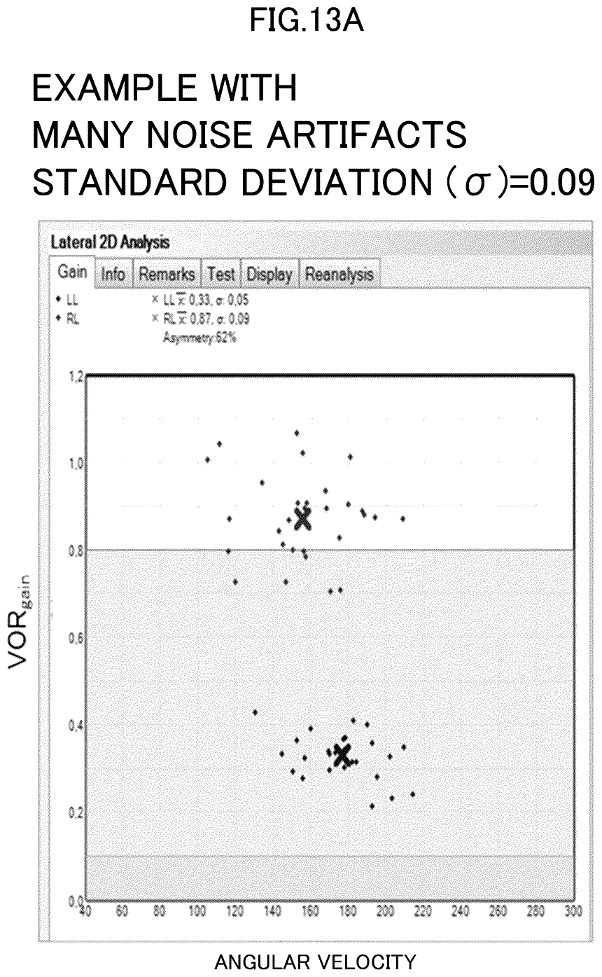

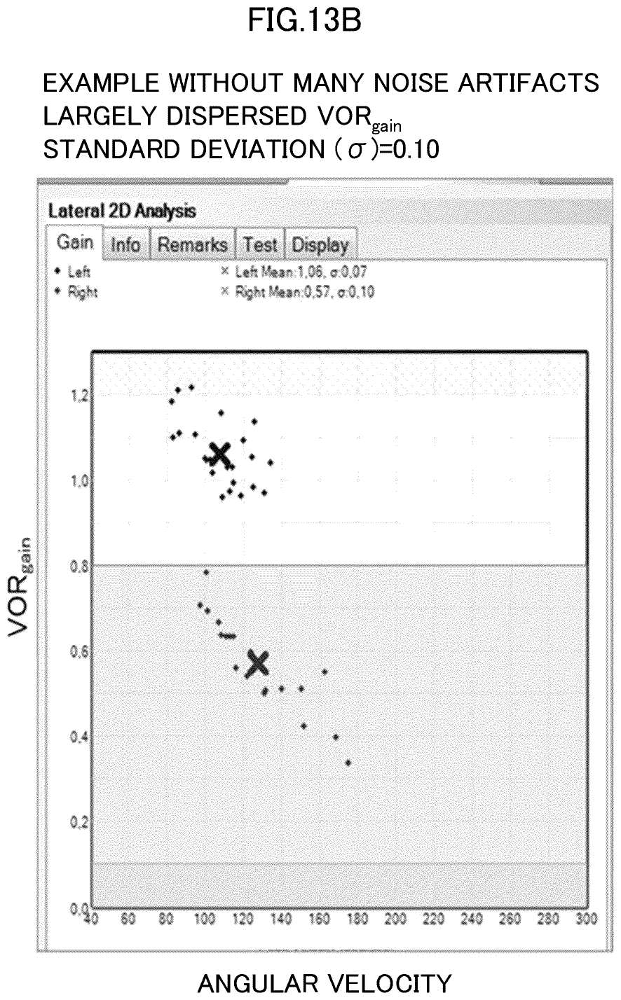

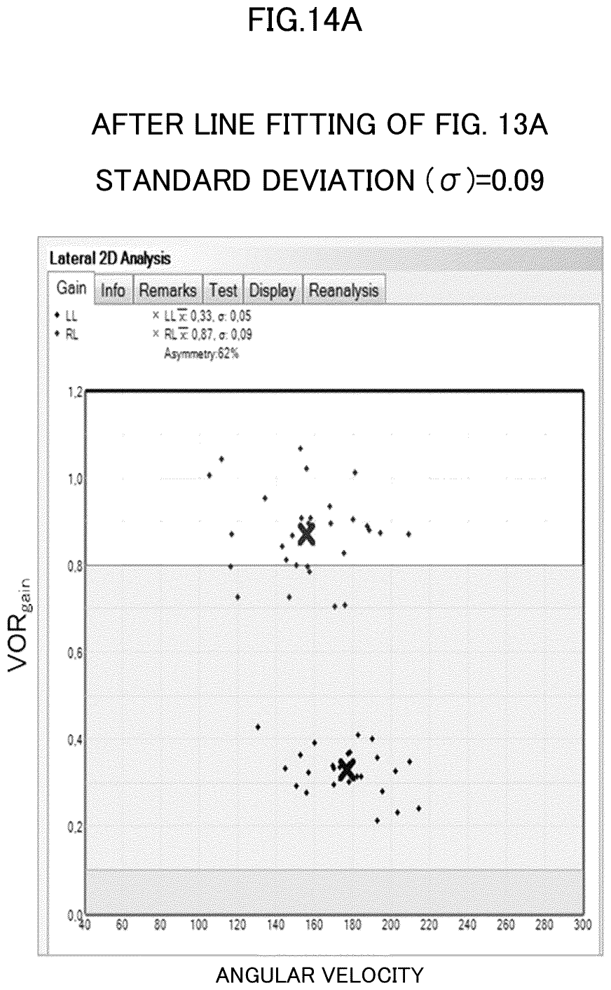

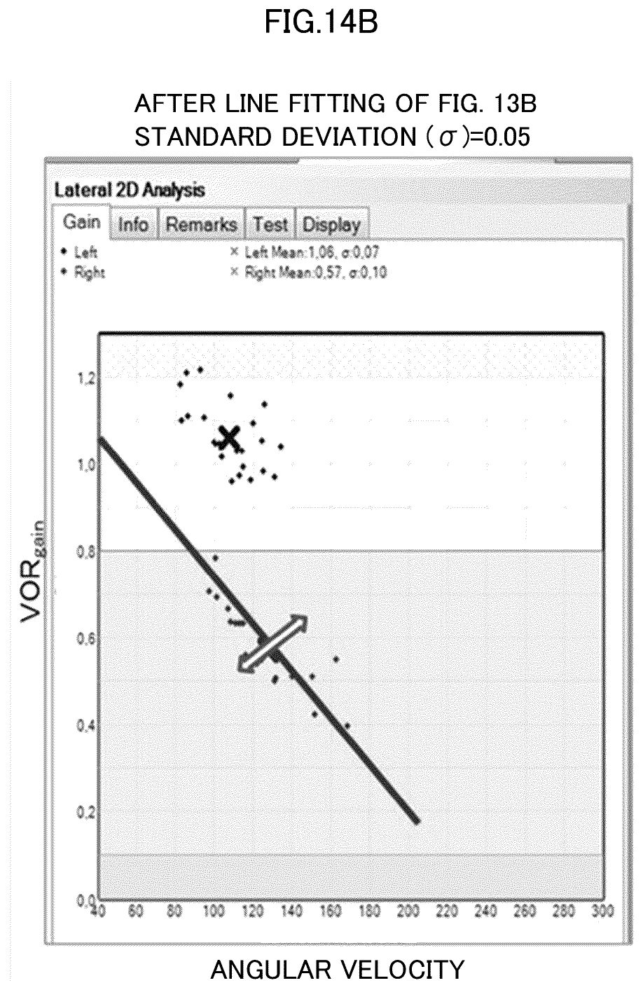

However, there are cases where the standard deviation (a) becomes small, i.e., 0.08, even when there are many noise artifacts, as depicted in FIG. 13A, and the standard deviation (a) becomes large, i.e., 0.10, even when there are not many noise artifacts, as depicted in FIG. 13B. Accordingly, there is a problem such that test accuracy cannot be accurately evaluated only with the standard deviation (a) of VOR_{gain}.

Therefore, a line of best fit for the results of each of FIGS. 13A and 13B is determined by line fitting according to the least squares method as presented in FIGS. 14A and 14B, and the accuracy is calculated from the line of best fit so that the test accuracy can be improved as presented in Table 1. Note that, the line of best fit includes a straight line of best fit and a curve of best fit.

Note that, the line of best fit obtained by the line fitting according to the least squares method can be determined by the equations (10), (12), and (14) presented above. Moreover, the calculations for aligning the arrow indicating the deviation in the scatter diagram of FIG. 14B were carried out by the equations (19) and (20) presented above.

TABLE 1

| | FIG. 13A | FIG. 14A | FIG. 13B | FIG. 14B |
|---|---|---|---|---|
| | Before line | After line | Before line | After line |
| | fitting | fitting | fitting | fitting |
| Stannard | 0.09 | 0.09 | 0.10 | 0.05 |
| deviation (σ) | | | | |

### Example 4

A test was performed on each of a patient A without slippage-induced artifacts, a patient B with slippage-induced artifacts, and a patient C to whom slippage-induced artifacts were caused at a constant rate every time by ISC Impulse (manufactured by Natus Medical Incorporated), in which the analysis program of the present invention was installed, in the same manner as in Example 1 to determine VOR_{gain} according to each of the direct method (angle ratio), the direct method (angular velocity ratio at 60 ms), and the indirect method VOR_{gain}. The difference in VOR_{gain} between the direct method and the indirect method was calculated to determine an absolute value of the difference between the direct method (angle ratio) and the indirect method, an absolute value of the difference between the direct method (angular velocity ratio at 60 ms) and the indirect method, and a standard deviation (o). The results of the patient A (the number of the tests performed: 10 times) are presented in Table 2, the results of the patient B (the number of the tests performed: 8 times) are presented in Table 3, and the results of the patient C (the number of the tests performed: 10 times) are presented in Table 4. The standard deviation (σ) is a value determined by the above-presented equation (16).

TABLE 2

| | | | | | Difference |
|---|---|---|---|---|---|
| | | | | Difference | (absolute value) |
| | | VOR_{gain} of | | (absolute value) | in VOR_{gain} between |
| | | direct method | | in VOR_{gain} between | direct method |
| | | (angular | | direct method | (angular |
| | VOR_{gain} of | velocity | VOR_{gain} of | (angle ratio) | velocity ratio) |
| | direct method | ratio at 60 | indirect | and indirect | and indirect |
| | (angle ratio) | ms) | method | method | method |
| 1 | 1.05 | 0.87 | 1.00 | 0.04 | 0.13 |
| 2 | 1.05 | 0.88 | 0.98 | 0.08 | 0.10 |
| 3 | 0.94 | 0.71 | 0.91 | 0.03 | 0.20 |
| 4 | 0.87 | 0.68 | 0.92 | 0.05 | 0.24 |
| 5 | 1.05 | 0.73 | 0.90 | 0.16 | 0.17 |
| 6 | 1.08 | 0.94 | 0.94 | 0.14 | 0.00 |
| 7 | 0.97 | 0.72 | 0.94 | 0.03 | 0.22 |
| 8 | 1.04 | 0.59 | 0.96 | 0.08 | 0.37 |
| 9 | 1.00 | 0.81 | 0.98 | 0.02 | 0.17 |
| 10 | 0.97 | 0.94 | 0.97 | 0.00 | 0.03 |
| Mean | 1.00 | 0.79 | 0.95 | 0.06 | 0.16 |
| Standard | 0.06 | 0.12 | 0.03 | 0.05 | 0.11 |
| deviation | | | | | |

TABLE 3

| | | | | | Difference |
|---|---|---|---|---|---|
| | | | | Difference | (absolute value) |
| | | VOR_{gain} of | | (absolute value) | in VOR_{gain} between |
| | | direct method | | in VOR_{gain} between | direct method |
| | | (angular | | direct method | (angular |
| | VOR_{gain} of | velocity | VOR_{gain} of | (angle ratio) | velocity ratio) |
| | direct method | ratio at 60 | indirect | and indirect | and indirect |
| | (angle ratio) | ms) | method | method | method |
| 1 | 0.72 | 0.97 | 0.49 | 0.22 | 0.48 |
| 2 | 0.78 | 0.72 | 0.43 | 0.35 | 0.29 |
| 3 | 0.77 | 1.23 | 0.60 | 0.17 | 0.63 |
| 4 | 0.87 | 1.05 | 0.59 | 0.28 | 0.46 |
| 5 | 1.10 | 0.63 | 0.61 | 0.49 | 0.02 |
| 6 | 0.81 | 1.43 | 0.49 | 0.32 | 0.94 |
| 7 | 0.91 | 1.33 | 0.54 | 0.37 | 0.79 |
| 8 | 0.61 | 1.53 | 0.43 | 0.18 | 1.10 |
| Mean | 0.82 | 1.11 | 0.52 | 0.30 | 0.59 |
| Standard | 0.15 | 0.33 | 0.07 | 0.11 | 0.35 |
| deviation | | | | | |

TABLE 4

| | | | | | Difference |
|---|---|---|---|---|---|
| | | | | Difference | (absolute value) |
| | | VOR_{gain} of | | (absolute value) | in VOR_{gain} between |
| | | direct method | | in VOR_{gain} between | direct method |
| | | (angular | | direct method | (angular |
| | VOR_{gain} of | velocity | VOR_{gain} of | (angle ratio) | velocity ratio) |
| | direct method | ratio at 60 | indirect | and indirect | and indirect |
| | (angle ratio) | ms) | method | method | method |
| 1 | 1.17 | 1.95 | 1.00 | 0.17 | 0.95 |
| 2 | 1.14 | 1.47 | 0.97 | 0.17 | 0.50 |
| 3 | 1.09 | 1.53 | 0.96 | 0.13 | 0.57 |
| 4 | 1.08 | 1.62 | 0.93 | 0.14 | 0.69 |
| 5 | 1.18 | 1.51 | 1.00 | 0.18 | 0.51 |
| 6 | 1.08 | 1.58 | 0.93 | 0.15 | 0.65 |
| 7 | 1.07 | 1.36 | 0.91 | 0.16 | 0.45 |
| 8 | 1.05 | 1.29 | 0.90 | 0.15 | 0.39 |
| 9 | 1.04 | 1.34 | 0.85 | 0.19 | 0.49 |
| 10 | 1.07 | 1.55 | 0.93 | 0.14 | 0.62 |
| Mean | 1.10 | 1.52 | 0.94 | 0.16 | 0.58 |
| Standard | 0.05 | 0.18 | 0.04 | 0.02 | 0.15 |
| deviation | | | | | |

It was found from the results of Table 2 that the patient A without slippage-induced artifacts had a small standard deviation with respect to all of the values of the direct method (angle ratio), the values of the indirect method, and the absolute values of the differences between the direct method (angle ratio) and the indirect method, and also had the small arithmetic mean of the differences between the (angle ratio) and the indirect method.

It was found from the results of Table 3 that the patient B with slippage-induced artifacts had a large standard deviation of the values of the direct method (angle ratio) and of the absolute values of the differences between the direct method (angle ratio) and the indirect method, but had a small standard deviation of the values of the indirect method. Moreover, it was found that the arithmetic mean of the differences between the direct method (angle ratio) and the indirect method was large.

It was found from the results of Table 4 that the patient C to whom slippage-induced artifacts were caused at a constant rate every time had a small standard deviation with respect to all of the values of the direct method (angle ratio), the value of the indirect method, and the absolute values of the difference between the direct method (angle ratio) and the indirect method, but the large arithmetic mean of the differences between the direct method (angle ratio) and the indirect method.

Accordingly, test accuracy can be appropriately evaluated corresponding to various levels of artifacts occurred by using the direct method and the indirect method in combination and determining both the arithmetic mean and standard deviation of the absolute values of the differences of the direct method and the indirect method. Therefore, test accuracy can be significantly improved compared to the technique of the related art.

### Example 5

VOR_{gain} of each patient of Cases 1 to 3 was determined by the direct method (angle ratio), the direct method (angular velocity ratio at 60 ms), and the indirect method in the same manner as in Example 1, using ICS Impulse (manufactured by Natus Medical Incorporated), in which the analysis program of the present invention was installed, and differences in the values between the direct method and the indirect method was calculated to determine the arithmetic mean and standard deviation (σ) of absolute values of the differences between the direct method (angle ratio) and the indirect method, and the arithmetic mean and standard deviation (σ) of absolute values of the differences between the direct method (angular velocity ratio at 60 ms) and the indirect method. The results are presented below.

### <Case 1>

TABLE 5

| | | | | | | Difference |
|---|---|---|---|---|---|---|
| | | | | | Difference | (absolute value) in |
| | | | VOR_{gain} of | | (absolute value) in | VOR_{gain} between |
| | | | direct | | VOR_{gain} between | direct method |
| | | VOR_{gain} of | method | | direct method | (angular |
| | | direct | (angular | VOR_{gain} of | (angle ratio) | velocity ratio) |
| | | method | velocity ratio | indirect | and indirect | and indirect |
| | | (angle ratio) | at 60 ms) | method | method | method |
| Without | Mean | 0.63 | 0.61 | 0.69 | 0.07 | 0.12 |
| slippage | Standard | 0.07 | 0.10 | 0.08 | 0.04 | 0.07 |
| | deviation | | | | | |
| With | Mean | 0.79 | 1.23 | 0.59 | 0.20 | 0.63 |
| slippage | Standard | 0.11 | 0.16 | 0.08 | 0.08 | 0.16 |
| | deviation | | | | | |

### <Case 2>

TABLE 6

| | | | | | | Difference |
|---|---|---|---|---|---|---|
| | | | | | Difference | (absolute value) in |
| | | | VOR_{gain} of | | (absolute value) in | VOR_{gain} between |
| | | | direct | | VOR_{gain} between | direct method |
| | | VOR_{gain} of | method | | direct method | (angular |
| | | direct | (angular | VOR_{gain} of | (angle ratio) | velocity ratio) |
| | | method | velocity ratio | indirect | and indirect | and indirect |
| | | (angle ratio) | at 60 ms) | method | method | method |
| Without | Mean | 0.57 | 0.62 | 0.54 | 0.06 | 0.07 |
| slippage | Standard | 0.05 | 0.07 | 0.05 | 0.05 | 0.04 |
| | deviation | | | | | |
| With | Mean | 0.80 | 1.11 | 0.54 | 0.26 | 0.44 |
| slippage | Standard | 0.16 | 0.33 | 0.06 | 0.13 | 0.29 |
| | deviation | | | | | |

### <Case 3>

TABLE 7

| | | | | | | Difference |
|---|---|---|---|---|---|---|
| | | | | | Difference | (absolute value) in |
| | | | VOR_{gain} of | | (absolute value) in | VOR_{gain} between |
| | | | direct | | VOR_{gain} between | direct method |
| | | VOR_{gain} of | method | | direct method | (angular |
| | | direct | (angular | VOR_{gain} of | (angle ratio) | velocity ratio) |
| | | method | velocity ratio | indirect | and indirect | and indirect |
| | | (angle ratio) | at 60 ms) | method | method | method |
| Without | Mean | 0.96 | 0.46 | 0.96 | 0.04 | 0.50 |
| slippage | Standard | 0.03 | 0.12 | 0.05 | 0.03 | 0.11 |
| | deviation | | | | | |
| With | Mean | 1.12 | 1.52 | 0.96 | 0.16 | 0.40 |
| slippage | Standard | 0.08 | 0.20 | 0.05 | 0.06 | 0.16 |
| | deviation | | | | | |

It can be understood from the results of Cases 1 to 3 that the results of the direct method (angular velocity ratio at 60 ms) have large standard deviations, and the data tends to vary, even without slippage-induced artifacts. Therefore, the direct method (angular velocity ratio at 60 ms) gives parameters that lack in stability compared to the direct method (angle ratio).

It can be understood from the results of Cases 1 and 2 that, if there are slippage-induced artifacts, the VOR_{gain} of the both direct methods (angle ratio) and (angular velocity ratio 60 ms) tends to have a large standard deviation. As an exception, Case 3 has relatively small standard deviations as the rate of the slippage is stable.

It is observed from the results of Cases 1 to 3 that both the arithmetic mean and the standard deviation of the differences between the direct method and the indirect method with slippage-induced artifacts become large compared to the case without slippage-induced artifacts.

It is observed from the results of Cases 1 to 3 that, when the slippage occurs at the constant rate every time, a standard deviation of the values of the differences between the direct method and the indirect method does not necessarily become large. Therefore, the parameter that can estimate test accuracy most suitably is considered to be the arithmetic mean of the differences in VOR_{gain} between the direct method (angle ratio) and the indirect method.

It is observed from the results of Cases 1 to 3 that the arithmetic mean and standard deviation of VOR_{gain} measured by the indirect method are stable regardless of the presence or absence of the slippage-induced artifacts.

### LIST OF REFERENCE SIGNS

100 analysis system
101 control unit
102 main memory device
103 auxiliary memory device
104 I/O interface
105 communication interface
106 input device
107 output device
108 display device

1. An analysis system for analysis of semicircular canal functions with rotational stimuli, the analysis system comprising:
a vestibulo ocular reflex data-acquiring unit configured to acquire first vestibulo ocular reflex data and second vestibulo ocular reflex data, where the first vestibulo ocular reflex data is acquired by dividing a rotation angle owing to a vestibulo ocular reflex with a rotation angle of a head position, and the second vestibulo ocular reflex data is acquired by subtracting at least a rotation angle owing to catch-up saccades (CUS) from the rotation angle of the head position to determine a residual rotation angle, followed by dividing the residual rotation angle with the rotation angle of the head position,
wherein the analysis system is configured to analyze semicircular canal functions based on the second vestibulo ocular reflex data, or based on both the first vestibulo ocular reflex data and the second vestibulo ocular reflex data.

2. The analysis system according to claim 1,
wherein the second vestibulo ocular reflex data is a value of VOR_{gain(I)} determined based on angular velocity data of a head of a subject and angular velocity data of eye movements of the subject, which are acquired by an angular velocity data-acquiring unit, according to the following equation:
VOR_{gain(I)}=(A−C)/A
wherein the angular velocity data-acquiring unit is set on the head of the subject and is configured to acquire the angular velocity data of the head and the angular velocity data of the eye movements when the head is turned, and
wherein, in the above equation, A is an integral of the angular velocity data of the head and C is an integral of the angular velocity data of the eyes of the subject owing to catch-up saccades (CUS) during a time when the head is turned.

3. The analysis system according to claim 2,
wherein the first vestibulo ocular reflex data is a value of VOR_{gain(D)} determined based on the angular velocity data of the head and the angular velocity data of the eye movements acquired by the angular velocity data-acquiring unit according to the following equation:
VOR_{gain(D)}=B/A
where A is the integral of the angular velocity data of the head, and B is an integral of the angular velocity data of the eye movements during the time when the head is turned.

4. The analysis system according to claim 1 further comprising:
an angular velocity data-acquiring unit that is set on a head of a subject and is configured to acquire angular velocity data of the head, and angular velocity data of eye movements of the subject when the head is turned; and
an analysis unit configured to determine, as the second vestibulo ocular reflex data, a value of VOR_{gain(I)} based on the angular velocity data of the head and the angular velocity data of the eye movements, which are acquired by the angular velocity data-acquiring unit, according to the following equation:
VOR_{gain(I)}=(A−C)/A
where A is an integral of the angular velocity data of the head and C is an integral of the angular velocity data of the eyes of the subject owing to catch-up saccades (CUS) during a time when the head is turned, and
to analyze semicircular canal functions of the subject based on the value of the VOR_{gain(I)}.

5. The analysis system according to claim 2,
wherein a data value of the A is a sum of a plurality of integrals of multiple angular velocity data of the head, and a data value of the C is a sum of a plurality of integrals of multiple angular velocity data of the eyes of the subject owing to the catch-up saccades (CUS).

6. The analysis system according to claim 2,
wherein the analysis system is configured to acquire a plurality of values of the VOR_{gain(1)}, determine a line of best fit from the acquired values of the VOR_{gain(I)}, and determine a value of the VOR_{gain(I)} at a certain angular velocity from the line of best fit.

7. The analysis system according to claim 2,
wherein the analysis system is configured to acquire a plurality of values of the VOR_{gain(I)}, determine a line of best fit from the acquired values of the VOR_{gain(I)}, and determine dispersion of the acquired values of the VOR_{gain(I)} from the line of best fit.

8. The analysis system according to claim 2,
wherein the analysis system is configured to determine values of VOR_{gain(D)} based on the angular velocity data of the head and the angular velocity data of the eye movements, which are acquired by the angular velocity data-acquiring unit, according to the following equation:
VOR_{gain(D)}=B/A
where A is an integral of the angular velocity data of the head and B is an integral of the angular velocity data of the eye movements during the time when the head is turned, and
the analysis system is configured to calculate an arithmetic mean and standard deviation of differences each between the value of the VOR_{gain (D)} and the value of the VOR_{gain (I)}.

9. The analysis system according to claim 8, further comprising:
a warning unit configured to display the values of VOR_{gain(D)}, the values of VOR_{gain(I)}, and the arithmetic mean and standard deviation of the differences each between the value of the VOR_{gain (D)} and the value of the VOR_{gain (I)}, and to issue a warning when the values exceed standard reference ranges.

10. The analysis system according to claim 2,
wherein the angular velocity data-acquiring unit includes a sensor and a camera, where the sensor is configured to collect angular velocity data associated with rotational movements of the head of the subject, and the camera is configured to capture the eye movements of the subject.

11. An analytical data acquisition device comprising:
a sensor configured to collect angular velocity data associated with rotational movements of a head of a subject; and
a camera configured to capture eye movements of the subject, wherein the analytical data acquisition device is used to analyze semicircular canal functions with rotational stimuli.

12. An analysis method for analysis of semicircular canal functions with rotational stimuli, the analysis method comprising:
a vestibulo ocular reflex data-acquiring process that includes
acquiring first vestibulo ocular reflex data and second vestibulo ocular reflex data,
where the first vestibulo ocular reflex data is acquired by dividing a rotation angle owing to a vestibulo ocular reflex with a rotation angle of a head position, and the second vestibulo ocular reflex data is acquired by subtracting at least a rotation angle owing to catch-up saccades (CUS) from the rotation angle of the head position to determine a residual rotation angle, followed by dividing the residual rotation angle with the rotation angle of the head position,
wherein the analysis method analyzes semicircular canal functions based on the second vestibulo ocular reflex data, or based on both the first vestibulo ocular reflex data and the second vestibulo ocular reflex data.

13. The analysis method according to claim 12, further comprising:
an angular velocity data-acquiring process that includes
acquiring angular velocity data of the head and angular velocity data of eye movements of a subject when the head is turned; and
an analysis process that includes
determining, as the second vestibulo ocular reflex data, a value of VOR_{gain(I)} based on the angular velocity data of the head and the angular velocity data of the eye movements, which are acquired in the angular velocity data-acquiring process, according to the following equation:
VOR_{gain(I)}=(A−C)/A
where A is an integral of angular velocity data of the head, and C is an integral of angular velocity data of the eyes of the subject owing to catch-up saccades (CUS) during a time when the head is turned, and
analyzing semicircular canal functions based on the value of the VOR_{gain(I)}.

14. (canceled)

15. (canceled)
